(19) United States
(12) Patent Application Publication
(10) Pub. No.: US 2021/0093298 A1
(43) Pub. Date: Apr. 1, 2021

(54) METHODS AND APPARATUSES FOR PROVIDING FEEDBACK FOR POSITIONING AN ULTRASOUND DEVICE

(71) Applicant: Butterfly Network, Inc., Guilford, CT (US)

(72) Inventors: Igor Lovchinsky, New York, NY (US); Yang Liu, Hoboken, NJ (US)

(73) Assignee: Butterfly Network, Inc., Guilford, CT (US)

(21) Appl. No.: 17/031,283

(22) Filed: Sep. 24, 2020

Related U.S. Application Data
(60) Provisional application No. 62/907,544, filed on Sep. 27, 2019.

(51) Int. Cl. A61B 8/08; A61B 8/00

(52) U.S. Cl. CPC A61B 8/523; A61B 8/4494; A61B 8/08; A61B 8/42

(57) ABSTRACT

Aspects of the technology described herein relate to providing feedback a user to position an ultrasound device. Some embodiments include configuring the ultrasound device to alternate collection of ultrasound images having one image plane and collection of ultrasound images having the other image plane, and providing feedback to the user to position the ultrasound device based on the ultrasound images. In such embodiments, feedback may be provided for simultaneously centering the anatomical structure in both types of ultrasound images. Some embodiments include collecting ultrasound images having one image plane, providing feedback for centering the anatomical structure in those ultrasound images, and once the anatomical structure is centered in those ultrasound images, collecting ultrasound images having another image plane and providing feedback for centering the anatomical structure in those ultrasound images.

### CROSS-REFERENCE TO RELATED APPLICATIONS

The present application claims the benefit under 35 U.S.C. § 119(e) of U.S. Patent Application Ser. No. 62/907,544, filed Sep. 27, 2019 under Attorney Docket No. B1348.70162US00, and entitled “METHODS AND APPARATUSES FOR PROVIDING FEEDBACK FOR POSITIONING AN ULTRASOUND DEVICE,” which is hereby incorporated by reference herein in its entirety.

### FIELD

Generally, the aspects of the technology described herein relate to providing feedback for collection of ultrasound images.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the application, a method for providing feedback to a user for positioning an ultrasound device includes: configuring, by a processing device in operative communication with the ultrasound device, the ultrasound device to collect one or more first ultrasound images having a first image plane and one or more second ultrasound images having a second image plane; and providing feedback to a user for positioning the ultrasound device based on the one or more first ultrasound images having the first image plane and/or the one or more second ultrasound images having the second image plane.

In some embodiments, configuring the ultrasound device to collect the one or more first ultrasound images having the first image plane and the one or more second ultrasound images having the second image plane includes configuring the ultrasound device to alternate collection of ultrasound images having the first image plane and ultrasound images having the second image plane. In some embodiments, configuring the ultrasound device to alternate collection of ultrasound images having the first image plane and ultrasound images having the second image plane includes configuring the ultrasound device to alternate the collection at a frame rate within a range of approximately 15-30 Hz. In some embodiments, configuring the ultrasound device to alternate collection of ultrasound images having the first image plane and ultrasound images having the second image plane includes configuring the ultrasound device to collect one ultrasound image having the first image plane and one ultrasound image having the second image plane. In some embodiments, configuring the ultrasound device to collect the one or more first ultrasound images having the first image plane and the one or more second ultrasound images having the second image plane includes configuring the ultrasound device to collect the one or more first ultrasound images and subsequently configuring the ultrasound device to collect the one or more second ultrasound images. In some embodiments, configuring the ultrasound device to collect the one or more first ultrasound images having the first image plane and the one or more second ultrasound images having the second image plane includes configuring the ultrasound device and/or the processing device to use beamforming to collect the one or more first ultrasound images having the first image plane and the one or more second ultrasound images having the second image plane.

In some embodiments, providing the feedback to the user for positioning the ultrasound device based on the one or more first ultrasound images having the first image plane and/or the one or more second ultrasound images having the second image plane includes providing feedback for simultaneously centering an anatomical structure depicted in the one or more first ultrasound images having the first image plane and the one or more second ultrasound images having the second image plane. In some embodiments, providing the feedback to the user for positioning the ultrasound device based on the one or more first ultrasound images having the first image plane and/or the one or more second ultrasound images having the second image plane includes providing feedback for centering an anatomical structure depicted in the one or more first ultrasound images having the first image plane and subsequently providing feedback for centering the anatomical structure depicted in the one or more second ultrasound images having the second image plane. In some embodiments, providing the feedback for centering the anatomical structure includes providing feedback for horizontally centering the anatomical structure. In some embodiments, providing the feedback for centering the anatomical structure includes providing feedback for vertically centering the anatomical structure.

In some embodiments, providing the feedback for simultaneously centering the anatomical structure depicted in the one or more first ultrasound images having the first image plane and the one or more second ultrasound images having the second image plane includes receiving a first ultrasound image having the first image plane, determining a first offset from center of the anatomical structure depicted in the first ultrasound image, receiving a second ultrasound image having the second image plane, determining a second offset from center of the anatomical structure depicted in the second ultrasound image, and providing feedback for positioning the ultrasound device based on the first and second offsets from center. In some embodiments, providing the feedback for positioning the ultrasound device based on the first and second offsets from center includes providing feedback for positioning the ultrasound device to minimize the first and second offsets from center.

In some embodiments, providing the feedback for centering the anatomical structure depicted in the one or more first ultrasound images having the first image plane and subsequently providing the feedback for centering the anatomical structure depicted in the one or more second ultrasound images having the second image plane includes receiving a first ultrasound image having the first image plane, determining a first offset from center of the anatomical structure depicted in the first ultrasound image, providing feedback for positioning the ultrasound device based on the first offset from center, and subsequent to providing the feedback for positioning the ultrasound device based on the first offset from center receiving a second ultrasound image having the second image plane, determining a second offset from center of the anatomical structure depicted in the second ultrasound image, and providing feedback for positioning the ultrasound device based on the second offset from center. In some embodiments, providing the feedback for positioning the ultrasound device based on the first offset from center includes providing feedback for positioning the ultrasound device to minimize the first offset from center. In some embodiments, providing the feedback for positioning the ultrasound device based on the second offset from center is performed subsequent to determining that the first offset from center is within a threshold of zero.

In some embodiments, determining the first offset from center includes determining a distance of the anatomical structure depicted in the first ultrasound image from a center portion of the first ultrasound image. In some embodiments, determining the distance of the anatomical structure depicted in the first ultrasound image from the center portion of the first ultrasound image includes determining the distance of a specific point on the anatomical structure depicted in the first ultrasound image from the center portion of the first ultrasound image, and wherein the specific point has predetermined mathematical characteristics. In some embodiments, the specific point includes a centroid of the anatomical structure. In some embodiments, the specific point includes a point on the anatomical structure that is farthest from all edge points of the anatomical structure.

In some embodiments, the method further includes automatically determining a location of the specific point on the anatomical structure. In some embodiments, the method further includes determining a location of the specific point on the anatomical structure using a statistical model. In some embodiments, the statistical model includes a segmentation model. In some embodiments, the statistical model includes a keypoint localization model. In some embodiments, the statistical model uses regression.

In some embodiments, determining the distance of the anatomical structure depicted in the first ultrasound image from the center portion of the first ultrasound image includes determining the distance of the anatomical structure depicted in the first ultrasound image from a vertical line positioned halfway across a horizontal dimension of the first ultrasound image. In some embodiments, determining the distance of the anatomical structure depicted in the first ultrasound image from the center portion of the first ultrasound image includes determining the distance of the anatomical structure depicted in the first ultrasound image from a horizontal line positioned halfway across a vertical dimension of the first ultrasound image.

In some embodiments, the feedback is of an implicit type. In some embodiments, the feedback is of an explicit type.

In some embodiments, providing the feedback for positioning the ultrasound device based on the first and second offsets from center includes displaying a marker, a horizontal line, and a vertical line such that a distance of the marker from the vertical line is proportional to the first offset from center and a distance of the marker from the horizontal line is proportional to the second offset from center. In some embodiments, providing the feedback for positioning the ultrasound device based on the first and second offsets from center includes displaying an arrow such that a length of a first component of the arrow is proportional to the first offset from center and a length of a second component of the arrow is proportional to the second offset from center. In some embodiments, providing the feedback for positioning the ultrasound device based on the first offset from center includes displaying a marker and a line such that a distance of the marker from the line is proportional to the first offset from center. In some embodiments, providing the feedback for positioning the ultrasound device based on the first offset from center includes displaying an arrow such that a length of the arrow is proportional to the first offset from center.

In some embodiments, the first image plane and the second image plane are orthogonal to each other. In some embodiments, the first image plane is along an azimuthal dimension of a transducer array of the ultrasound device, and the second image plane is along an elevational dimension of the transducer array of the ultrasound device. In some embodiments, the first image plane is along an elevational dimension of a transducer array of the ultrasound device, and the second image plane is along an azimuthal dimension of the transducer array of the ultrasound device.

In some embodiments, the anatomical structure includes a bladder. In some embodiments, the method further includes performing an ultrasound imaging sweep subsequent to determining that the first and second offsets from center are within a threshold of zero.

According to another aspect of the application, a method for changing an imaging depth of an ultrasound device includes: receiving, by a processing device in operative communication with the ultrasound device, an ultrasound image; determining an offset from center of an anatomical structure depicted in the ultrasound image; and providing feedback for changing the imaging depth of the ultrasound device based on the offset from center or automatically changing the imaging depth of the ultrasound device based on the offset from center.

In some embodiments, the offset from center includes a horizontal offset from center. In some embodiments, the offset from center includes a vertical offset from center.

In some embodiments, providing the feedback for changing the imaging depth of the ultrasound device based on the offset from center includes providing feedback for changing the imaging depth of the ultrasound device to minimize the offset from center. In some embodiments, automatically changing the imaging depth of the ultrasound device based on the offset from center includes automatically changing the imaging depth of the ultrasound device to minimize the offset from center.

In some embodiments, the feedback is of an implicit type. In some embodiments, the feedback is of an explicit type.

In some embodiments, determining the offset from center includes determining a distance of the anatomical structure depicted in the ultrasound image from a center portion of the ultrasound image. In some embodiments, determining the distance of the anatomical structure depicted in the first ultrasound image from the center portion of the ultrasound image includes determining the distance of a specific point on the anatomical structure depicted in the ultrasound image from the center portion of the ultrasound image, and wherein the specific point has predetermined mathematical characteristics. In some embodiments, the specific point includes a centroid of the anatomical structure. In some embodiments, the specific point includes a point on the anatomical structure that is farthest from all edge points of the anatomical structure.

In some embodiments, the method further includes automatically determining a location of the specific point on the anatomical structure. In some embodiments, the method further includes determining a location of the specific point on the anatomical structure using a statistical model. In some embodiments, the statistical model includes a segmentation model. In some embodiments, the statistical model includes a keypoint localization model. In some embodiments, the statistical model uses regression.

In some embodiments, determining the distance of the anatomical structure depicted in the ultrasound image from the center portion of the ultrasound image includes determining the distance of the anatomical structure depicted in the ultrasound image from a vertical line positioned halfway across a horizontal dimension of the ultrasound image. In some embodiments, determining the distance of the anatomical structure depicted in the ultrasound image from the center portion of the ultrasound image includes determining the distance of the anatomical structure depicted in the ultrasound image from a horizontal line positioned halfway across a vertical dimension of the ultrasound image.

In some embodiments, providing the feedback for changing the imaging depth of the ultrasound device based on the offset from center includes displaying a marker and a line such that a distance of the marker from the line is proportional to the offset from center. In some embodiments, providing the feedback for changing the imaging depth of the ultrasound device based on the offset from center includes displaying an indication as to whether the imaging depth of the ultrasound device should be increased or decreased.

In some embodiments, the anatomical structure includes a bladder. In some embodiments, the method further includes performing an ultrasound imaging sweep subsequent to determining that the offset from center is within a threshold of zero.

Some aspects include at least one non-transitory computer-readable storage medium storing processor-executable instructions that, when executed by at least one processor, cause the at least one processor to perform the above aspects and embodiments. Some aspects include a method to perform the actions that the processing device is configured to perform.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates a process for providing feedback to a user for positioning an ultrasound device, in accordance with certain embodiments described herein;

FIG. 2 illustrates example image planes with respect to a subject, in accordance with certain embodiments described herein;

FIG. 3 illustrates an example ultrasound image, in accordance with certain embodiments described herein;

FIG. 4 illustrates another example ultrasound image, in accordance with certain embodiments described herein;

FIG. 5 illustrates an example feedback display for positioning an ultrasound device, in accordance with certain embodiments described herein;

FIG. 6A illustrates another example feedback display for positioning an ultrasound device, in accordance with certain embodiments described herein;

FIG. 6B illustrates another example feedback display for positioning an ultrasound device, in accordance with certain embodiments described herein;

FIG. 7 illustrates another process for providing feedback to a user for positioning an ultrasound device, in accordance with certain embodiments described herein;

FIG. 8 illustrates another example feedback display for positioning an ultrasound device, in accordance with certain embodiments described herein;

FIG. 9 illustrates another example feedback display for positioning an ultrasound device, in accordance with certain embodiments described herein;

FIG. 10 illustrates another example feedback display for positioning an ultrasound device, in accordance with certain embodiments described herein;

FIG. 11A illustrates another example feedback display for positioning an ultrasound device, in accordance with certain embodiments described herein;

FIG. 11B illustrates another example feedback display for positioning an ultrasound device, in accordance with certain embodiments described herein;

FIG. 11C illustrates another example feedback display for positioning an ultrasound device, in accordance with certain embodiments described herein;

FIG. 12 illustrates a process for providing feedback to a user for changing the imaging depth of an ultrasound device, in accordance with certain embodiments described herein;

FIG. 13 illustrates an example ultrasound image, in accordance with certain embodiments described herein;

FIG. 14 illustrates another example feedback display for positioning an ultrasound device, in accordance with certain embodiments described herein;

FIG. 15A illustrates another example feedback display for positioning an ultrasound device, in accordance with certain embodiments described herein;

FIG. 15B illustrates another example feedback display for positioning an ultrasound device, in accordance with certain embodiments described herein;

FIG. 15C illustrates a process for automatically changing the imaging depth of an ultrasound device, in accordance with certain embodiments described herein;

FIG. 16 illustrates a schematic block diagram of an example ultrasound system upon which various aspects of the technology described herein may be practiced.

### DETAILED DESCRIPTION

Ultrasound imaging sweeps may be useful for certain applications. For example, an ultrasound imaging sweep may be used for collecting three-dimensional ultrasound data for measuring the volume of an anatomical structure and/or for generating a three-dimensional visualization of an anatomical structure. An ultrasound imaging sweep may include generating multiple ultrasound beams focused along one direction, each steered at a different angle relative to a different direction. For example, an ultrasound imaging sweep may include generating multiple ultrasound beams focused along the azimuthal dimension of an ultrasound device's transducer array, each of these beams steered at a different angle relative to the elevational dimension of the transducer array. In other words, ultrasound beams focused along the azimuthal dimension may be swept along the elevational dimension.

When performing such sweeps, it may be helpful to guide a user to position the ultrasound device and change the imaging depth such that the anatomical structure is centered with respect to the transducer array (e.g., with respect to both the azimuthal and elevational dimensions of the transducer array) and with respect to the depth dimension of the field of view of the ultrasound device. In other words, it may be helpful to guide a user to position the ultrasound device and change the imaging depth such that the anatomical structure is horizontally and vertically centered in ultrasound images collected during the ultrasound sweep. This may help improve the accuracy of calculations performed based on the anatomical structure in ultrasound images collected during the ultrasound sweep, as centering the anatomical structure may help to minimize the chance that a portion of the anatomical structure will be outside the range of the ultrasound sweep. Additionally, the quality of data in ultrasound images generated from the sweep may be better in the center of the images.

The inventors have recognized that to guide a user to position the ultrasound device such that the anatomical structure is centered with respect to the transducer array of the ultrasound device, it may be helpful to collect ultrasound images having image planes along the azimuthal and elevational dimensions of the transducer array and provide feedback for positioning the ultrasound device to center (e.g., horizontally) an anatomical structure in both types of images. Some embodiments for guiding the user may include configuring the ultrasound device to alternate collection of ultrasound images having one image plane and collection of ultrasound images having the other image plane. (As referred to herein, alternating collection of ultrasound images having one image plane and collection of ultrasound images having the other image plane includes collecting just one ultrasound image having each image plane.) In such embodiments, feedback may be provided for positioning the ultrasound device to simultaneously center the anatomical structure in both types of ultrasound images. Some embodiments for guiding the user may include configuring the ultrasound device to collect ultrasound images having one image plane and then configuring the ultrasound device to collect ultrasound images having another image plane. In such embodiments, feedback may be provided for positioning the ultrasound device to center the anatomical structure in the ultrasound images having the first image plane, and once the anatomical structure is centered in those ultrasound images, feedback may be provided for positioning the ultrasound device to center the anatomical structure in the ultrasound images having the second image plane. More generally, some embodiments for guiding user may include configuring the ultrasound device to collect ultrasound images having two or more image planes in three-dimensional space, and providing feedback for positioning the ultrasound device to center an anatomical structure in all the types of images. Feedback may be of an explicit type, in which the feedback may explicitly instruct the user how to move the ultrasound device, and/or feedback may be of an implicit type, in which the feedback may indicate the current position of the ultrasound device, and the user may determine based on the current position of the ultrasound device how to move the ultrasound device.

The inventors have recognized that to guide a user to position the ultrasound device such that the anatomical structure is centered with respect to the depth dimension of the field of view of the ultrasound device, it may be helpful to collect one or more ultrasound images and provide feedback for changing the imaging depth to center (e.g., vertically) an anatomical structure in the one or more ultrasound images. Feedback may be of an explicit type, in which the feedback may explicitly instruct the user how to change the imaging depth, and/or feedback may be of an implicit type, in which the feedback may indicate the current position of the anatomical structure in ultrasound images, and the user may determine based on the current position of the anatomical structure in the ultrasound images how to change the imaging depth. In some embodiments, rather than providing feedback to a user to change the imaging depth, the ultrasound device may be automatically configured with a particular imaging depth such that the anatomical structure is centered with respect to the depth dimension of the field of view of the ultrasound device.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

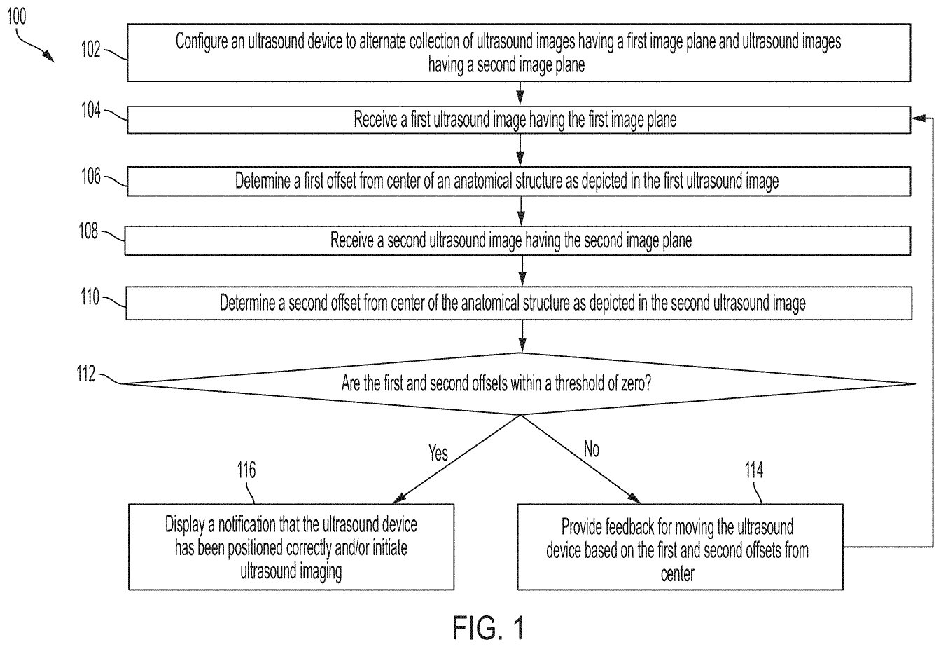

FIG. 1 illustrates a process 100 for providing feedback to a user for positioning an ultrasound device, in accordance with certain embodiments described herein. The process 100 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself (of which the processing device may be a part) may perform the process 100. The process 100 is directed to instructing a user to center an anatomical structure with respect to the transducer array of an ultrasound device.

In act 102, the processing device configures an ultrasound device to alternate collection of ultrasound images having a first image plane and ultrasound images having a second image plane. In some embodiments, the processing device may configure the ultrasound device to collect an ultrasound image having the first image plane, then an ultrasound image having the second image plane, then an ultrasound image having the first image plane, then an ultrasound image having the second image plane, etc. For example, the processing device may configure the ultrasound device to alternate collection of the ultrasound images at a rate in the range of approximately 15-30 Hz. In some embodiments, the first and second image planes may be orthogonal to each other. In some embodiments, the first image plane may be along the azimuthal dimension of the ultrasound device's transducer array, and the second image plane may be along the elevational dimension of the ultrasound device's transducer array, or vice versa. In some embodiments, one or more of the image planes may extend diagonally across the ultrasound device's transducer array. It should be appreciated that these are non-limiting examples of image planes. In some embodiments, other image planes may be used, and the image planes need not be orthogonal to each other. The processing device may configure the ultrasound device and/or may configure itself to use beamforming to focus collection of the ultrasound images along the particular directions. The process 100 proceeds from act 102 to act 104.

In act 104, the processing device receives a first ultrasound image having the first image plane. The first ultrasound image may have been collected when the ultrasound device was configured by the processing device to collect ultrasound images having the first image plane. The first ultrasound image may be generated based on raw ultrasound data collected by the ultrasound device. In some embodiments, the ultrasound device may generate the ultrasound image based on the raw ultrasound data, and at act 104, the processing device may receive the ultrasound image from the ultrasound device. In some embodiments, the ultrasound device may generate scan lines from the raw ultrasound data, and at act 104, the processing device may receive the scan lines from the ultrasound device and generate the ultrasound image based on the scan lines. In some embodiments, at act 104, the processing device may receive the raw ultrasound data from the ultrasound device and generate the ultrasound image based on the raw ultrasound data. The ultrasound image may be the most recently collected ultrasound image by the ultrasound device, and the processing device may receive the ultrasound image in real-time, as it is collected. The process 100 proceeds from act 104 to act 106.

In act 106, the processing device determines a first offset from center of an anatomical structure as depicted in the first ultrasound image. The first offset from center may correspond to a distance of the anatomical structure (e.g., a bladder) depicted in the first ultrasound image from a center portion of the first ultrasound image. In some embodiments, the processing device may measure the distance from a specific point on the anatomical structure, where the point has predetermined mathematical characteristics, to the center portion of the first ultrasound image. Examples of the specific point include the centroid of the anatomical structure and the point on the anatomical structure that is farthest from all the edge points of the anatomical structure, although other specific points may be used. In some embodiments, the processing device may automatically determine the location of the specific point on the anatomical structure. In some embodiments, a statistical model may be trained to determine the location of a specific point on an anatomical structure depicted in an ultrasound image. The statistical model may be stored on the processing device or stored on another electronic device (e.g., a server) and accessed by the processing device.

For the example where the specific point is the centroid of the anatomical structure, in some embodiments, the statistical model may be trained on multiple pairs of input and output training data sets as a segmentation model. Each set of input training data may be an ultrasound image depicting an anatomical structure. Each set of output training data may be a segmentation mask that is an array of values equal in size to the input training data ultrasound image, and pixels corresponding to locations within the anatomical structure in the ultrasound image are manually set to 1 and other pixels are set to 0 (although other values may be used instead). Based on this training data, the statistical model may learn to output, based on an inputted ultrasound image, a segmentation mask where each pixel has a value representing the probability that the pixel corresponds to a location within the anatomical structure in the ultrasound image (values closer to 1) or outside the anatomical structure (values closer to 0). The processing device may select all pixels in the segmentation mask that have a value greater than a threshold value (e.g., 0.5) as being within the anatomical structure. To determine the location of the centroid of the anatomical structure depicted in the ultrasound image, the processing device may calculate the arithmetic mean of all the locations of pixels that were determined to be within the anatomical structure. For example, the processing device may calculate the arithmetic mean of the horizontal locations of all pixels within the anatomical structure and the arithmetic mean of the vertical locations of all pixels within the anatomical structure. The processing device may determine the location of the centroid of the anatomical structure to be the pixel having a horizontal position that is at the arithmetic mean of all pixels within the anatomical structure and having a vertical position that is at the arithmetic mean of all pixels within the anatomical structure.

In some embodiments, the statistical model may be trained on multiple pairs of input and output training data sets as a keypoint localization model. Each set of input training data may be an ultrasound image depicting an anatomical structure. Each set of output training data may be an array of values that is the same size as the input training data ultrasound image, where the pixel corresponding to the centroid of the anatomical structure in the ultrasound image is manually set to a value of 1 and every other pixel has a value of 0 (although other values may be used instead). Based on this training data, the statistical model may learn to output, based on an inputted ultrasound image, an array of values that is the same size as the inputted image, where each pixel in the array consists of a probability that that pixel is where the centroid of an anatomical structure depicted in the ultrasound image is located. The processing device may select the pixel having the highest probability as the location of the specific point on the anatomical structure in the ultrasound image.

In some embodiments, the statistical model may be trained on multiple pairs of input and output training data sets to use regression. Each set of input training data may be an ultrasound image depicting an anatomical structure. Each set of output training data may be the pixel location of the centroid of the anatomical structure in the input training data ultrasound image. Based on this training data, the statistical model may learn to output, based on an inputted ultrasound image, the horizontal and vertical pixel coordinates of the centroid of an anatomical structure depicted in the ultrasound device.

For the example where the specific point is the point on the anatomical structure that is farthest from all the edge points of the anatomical structure, in some embodiments, the statistical model may be trained on multiple pairs of input and output training data sets as a segmentation model. Each set of input training data may be an ultrasound image depicting an anatomical structure. Each set of output training data may be a segmentation mask that is an array of values equal in size to the input training data ultrasound image, and pixels corresponding to locations on the boundary of the anatomical structure in the ultrasound image are manually set to 1 and other pixels are set to 0 (although other values may be used instead). Based on this training data, the statistical model may learn to output, based on an inputted ultrasound image, a segmentation mask where each pixel has a value representing the probability that the pixel corresponds to a boundary of the anatomical structure in the ultrasound image (values closer to 1) or does not correspond to a boundary of the anatomical structure (values closer to 0). The processing device may select all pixels in the segmentation mask that have a value greater than a threshold value (e.g., 0.5) as being on the boundary of the anatomical structure. To determine the location of the point on the anatomical structure that is farthest from all the edge points of the anatomical structure depicted in the ultrasound image, the processing device may calculate, for every pixel inside the boundary, the sum of the distances of that pixel to every pixel on the boundary. The processing device may then select the pixel having the greater sum of distances as the location of the specific point on the anatomical structure in the ultrasound image.

In some embodiments, the statistical model may be trained on multiple pairs of input and output training data sets as a keypoint localization model. Each set of input training data may be an ultrasound image depicting an anatomical structure. Each set of output training data may be an array of values that is the same size as the input training data ultrasound image, where the pixel corresponding to the point on the anatomical structure that is farthest from all the edge points of the anatomical structure in the ultrasound image is manually set to a value of 1 and every other pixel has a value of 0 (although other values may be used instead). Based on this training data, the statistical model may learn to output, based on an inputted ultrasound image, an array of values that is the same size as the inputted image, where each pixel in the array consists of a probability that that pixel is where the point on the anatomical structure that is farthest from all the edge points of the anatomical structure in the ultrasound image is located. The processing device may select the pixel having the highest probability as the location of the specific point on the anatomical structure in the ultrasound image.

In some embodiments, the statistical model may be trained on multiple pairs of input and output training data sets to use regression. Each set of input training data may be an ultrasound image depicting an anatomical structure. Each set of output training data set may be the pixel location of the point on the anatomical structure that is farthest from all the edge points of the anatomical structure. Based on this training data, the statistical model may learn to output, based on an inputted ultrasound image, the horizontal and vertical pixel coordinates of the point on the anatomical structure that is farthest from all the edge points of the anatomical structure.

In some embodiments, the center portion of the first ultrasound image may be a vertical line positioned halfway across a horizontal dimension of the first ultrasound image, and the processing device may measure the horizontal distance from the specific point on the anatomical structure to the vertical line. This may be the case if the horizontal dimension of the first ultrasound image is parallel to the plane of the transducer array of the ultrasound device. In some embodiments, the center portion of the first ultrasound image may be a horizontal line positioned halfway across a vertical dimension of the first ultrasound image, and the processing device may measure the vertical distance from the specific point on the anatomical structure to the horizontal line. This may be the case if the vertical dimension of the first ultrasound image is parallel to the plane of the transducer array of the ultrasound device. This distance may be the first offset from center determined in act 106. The process 100 proceeds from act 106 to act 108.

In act 108, the processing device receives a second ultrasound image having the second image plane. The second ultrasound image may have been collected when the ultrasound device was configured by the processing device to collect ultrasound images having the second image plane. In some embodiments, if the first image plane was along the azimuthal dimension of the ultrasound device's transducer array, then the second image plane may be along the elevational dimension of the ultrasound device's transducer array, and vice versa. Further description of receiving ultrasound images may be found with reference to act 104. The process 100 proceeds from act 108 to act 110.

In act 110, the processing device determines a second offset from center of the anatomical structure as depicted in the second ultrasound image. Further description of determining offsets from center may be found with reference to act 106. The process 100 proceeds from act 110 to act 112.

In act 112, the processing device determines if the first and second offsets are both within a threshold of zero. For example, the processing device may determine if the specific point on the anatomical structure in both the first and second ultrasound images is within a threshold distance of a vertical line positioned halfway across the horizontal dimension of the images. If no, then the processing device proceeds from act 112 to act 114. If yes, the process 100 proceeds from act 112 to act 116. In act 116 the processing device displays a notification that the ultrasound device has been positioned correctly and/or initiates (e.g., automatically) ultrasound imaging (e.g., an ultrasound imaging sweep). The process 100 may then terminate. In some embodiments, the processing device may perform another action based on determining, in act 112, that the first and second offsets are both within a threshold of zero. Thus, in some embodiments, act 116 may be omitted.

In some embodiments, if the processing device determines that the first offset from center is within a threshold of zero but the second offset from center is not, the processing device may configure the ultrasound device to collect ultrasound images having the second image plane (if it is not so configured already) and proceed to act 108. In other words, the processing device may configure the ultrasound device to only collect ultrasound images having the second image plane, and not the first image plane. In some embodiments, if the processing device determines that the second offset from center is within a threshold of zero but the first offset from center is not, the processing device may configure the ultrasound device to collect ultrasound images having the first image plane (if it is not so configured already), proceed to act 104, and after act 106, proceed to act 114. In other words, the processing device may configure the ultrasound device to only collect ultrasound images having the first image plane, and not the second image plane. In such embodiments, the processing device may provide an instruction not to move the ultrasound device in a direction that may undo the centering in the already-centered image plane. For example, if the first image plane is along the left-right dimension of the subject, and the processing device determines that the first offset from center is within a threshold of zero, then when the processing device configures the ultrasound device to only collect ultrasound images having the second image plane, the processing device may provide an instruction not to move the ultrasound device along the left-right dimension of the subject.

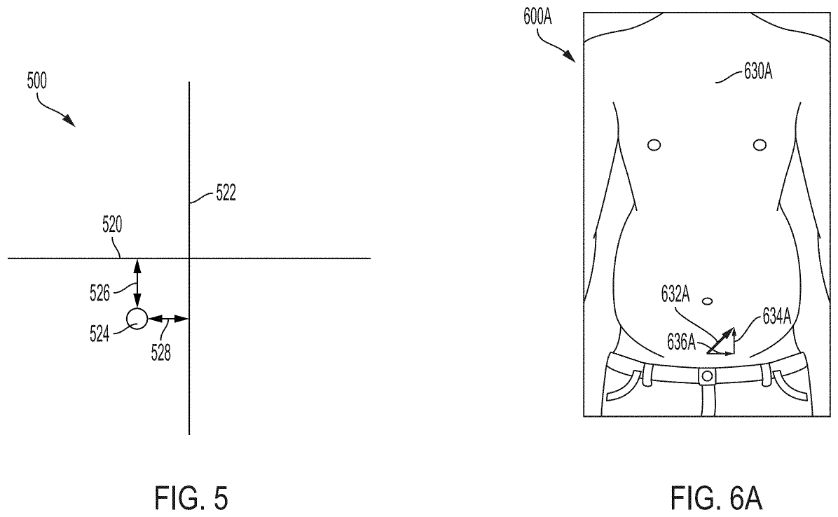

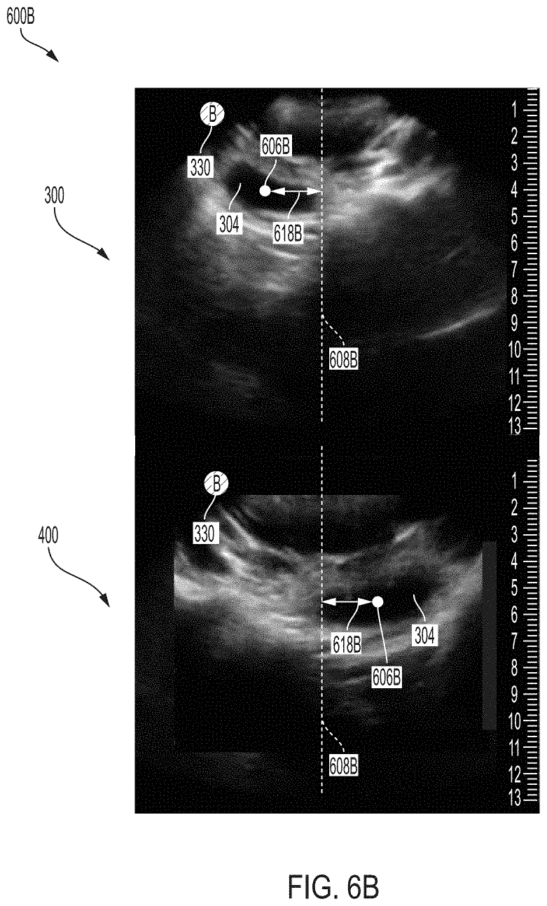

In act 114, the processing device provides feedback for positioning the ultrasound device based on the first and second offsets from center determined in acts 106 and 110. In some embodiments, the feedback may be to move the ultrasound device to minimize the first and second offsets from center. For example, consider an example in which the first and second offsets from center quantify a distance on an ultrasound image from a specific point on an anatomical structure to a vertical line on the ultrasound image positioned halfway across the horizontal dimension of the ultrasound image. The feedback may be to move the ultrasound device such that subsequent ultrasound images having the first image plane depict the anatomical structure closer to the vertical line, and subsequent ultrasound images having the second image plane depict the anatomical structure closer to the vertical line. In some embodiments, the feedback may be of an implicit type, in which the feedback may indicate the current position of the ultrasound device, and the user may determine based on the current position of the ultrasound device how to move the ultrasound device. For example, the feedback may indicate that the ultrasound device is too far to the left relative to the subject. In such embodiments, the feedback may not include an explicit instruction to move the ultrasound device in a particular manner. In some embodiments, the feedback may be of an explicit type, in which the feedback may explicitly instruct the user how to move the ultrasound device. For example, the feedback may indicate that the ultrasound device should be moved rightwards relative to the subject. Further description of such feedback may be found with reference to FIGS. 5-6B, described in further detail hereinafter. If either the first or second offsets from center is within a threshold of zero, in some embodiments, the processing device may only provide feedback for positioning the ultrasound device based on the offset from center than is not within a threshold zero. The process 100 proceeds from act 114 to act 104. This may be the case in embodiments in which, at act 102, the processing device configures the ultrasound device to alternate collection of ultrasound images having the first image plane and having the second image plane indefinitely, or for an extended period of time. In some embodiments, at act 102, the processing device may configure the ultrasound device to alternate collection of one ultrasound image having the first image plane and one ultrasound image having the second image plane. In such embodiments, after act 114, the processing device may return to act 102.

In some embodiments, act 102 may be omitted, for example, if the ultrasound device has been already configured to collect ultrasound images in this way. In some embodiments, act 112 may be omitted. In such embodiments, if the first and second offsets are both within a threshold of zero, the feedback provided at act 114 may be to not move the ultrasound device.

In some embodiments, instead of configuring the ultrasound device to alternate collection of ultrasound images having the first image plane and having the second image plane at act 102, the processing device may configure the ultrasound device to collect an ultrasound image having the first image plane prior to act 104, and configure the ultrasound device to collect an ultrasound image having the second image plane prior to act 108.

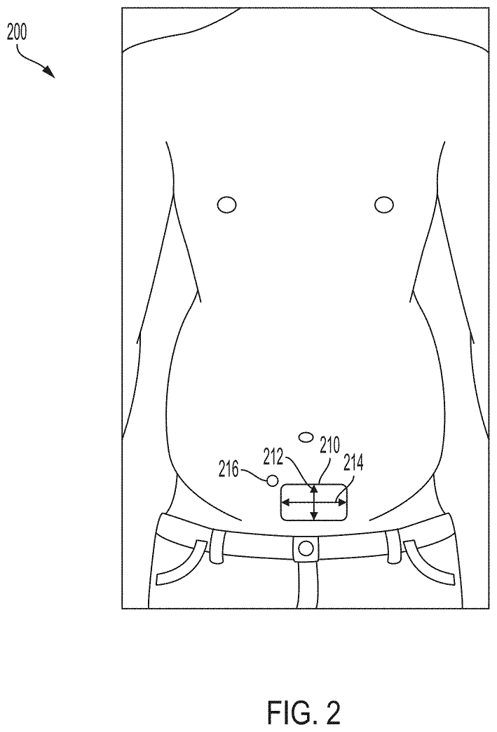

FIG. 2 illustrates example image planes with respect to a subject 200, in accordance with certain embodiments described herein. FIG. 2 includes the subject 200, a footprint of a transducer array 210 of an ultrasound device when the ultrasound device is placed on the subject 200, an elevational dimension 212 of the transducer array 210, an azimuthal dimension 214 of the transducer array 210, and an orientation indicator 216. In some embodiments, the first image plane may be along the elevational dimension 212 of the transducer array 210, and the second image plane may be along the azimuthal dimension 214 of the transducer array 210, or vice versa. In FIG. 2, the azimuthal dimension 214 of the transducer array 210 is along the left-right dimension of the subject 200 and the elevational dimension 212 of the subject 200 is along the up-down dimension of the subject 200. However, the transducer array 210 may be oriented in other orientations relative to the subject. For example, the azimuthal dimension 214 of the transducer array 210 may be along the up-down dimension of the subject 200 and the elevational dimension 212 of the subject 200 may be along the right-left dimension of the subject 200.

The ultrasound device may include a physical marker adjacent to a portion of its transducer array 210. Ultrasound images collected by the ultrasound device may include an orientation indicator displayed on either the left or right side of the ultrasound images. Portions of the ultrasound images closer to the orientation indicator may depict data collected by portions of the transducer array 210 closer to the physical marker, and portions of the ultrasound images farther from the orientation indicator may depict data collected by portions of the transducer array 210 farther from the physical marker. The orientation indicator 216 corresponds to an example location of a physical marker relative to the transducer array 210.

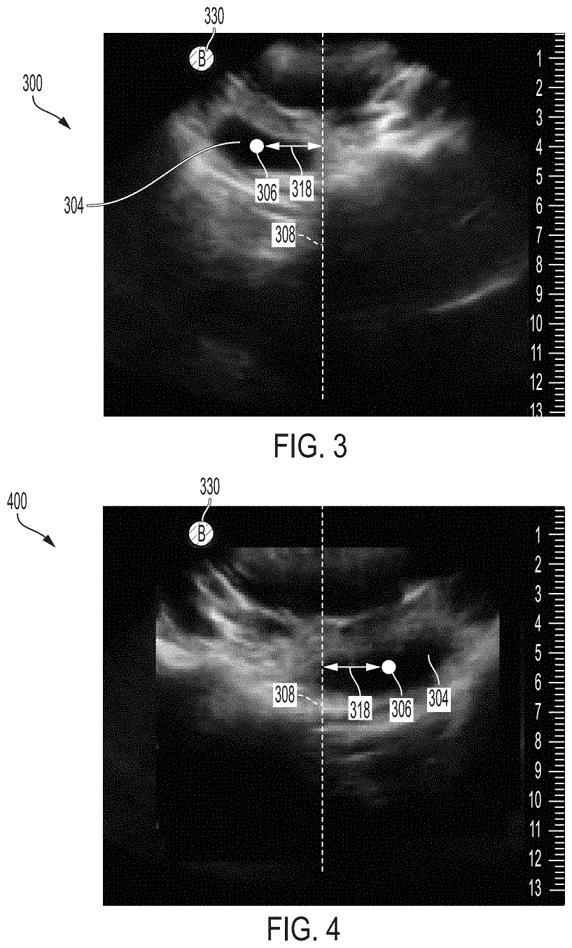

FIG. 3 illustrates an example ultrasound image 300, in accordance with certain embodiments described herein. The ultrasound image 300 depicts an anatomical structure 304 (in the example of FIG. 3, a bladder), and includes an orientation indicator 330. The orientation indicator 330 is positioned on the left of the ultrasound image 300 and the ultrasound image 400 in FIG. 4, but may be positioned on the right of the ultrasound images. FIG. 3 further illustrates a specific point 306 on the anatomical structure 304, a vertical line 308, and a distance 318. The ultrasound image 300 may have an image plane along, for example, either the elevational dimension 212 or the azimuthal dimension 214 of the transducer array 210. The specific point may have predetermined mathematical characteristics, and the location of the specific point 306 may have been automatically calculated by a processing device. For example, the specific point 306 may be the centroid of the anatomical structure 304 or the point that is farthest from all the edges of the anatomical structure 304. The vertical line 308 is positioned halfway across the horizontal dimension of the ultrasound image 300. The distance 318 is the horizontal distance from the specific point 306 to the vertical line 308. Consider, as an example, that the ultrasound image was collected along the azimuthal dimension 214 of the transducer array 210. The distance 318 indicates that the specific point 306 on the anatomical structure 304 is to the left of the vertical line 308 in the ultrasound image 300. The position of the orientation indicator 330 to the left of the ultrasound image 300 and the position of the orientation indicator 216 on the left side of the subject 200 relative to the transducer array 210 of the ultrasound device indicate that leftwards in the ultrasound image 300 corresponds to leftwards relative to the subject 200. Thus, the ultrasound device may be too far to leftwards on the subject 200 and may need to be moved rightwards on the subject 200 such that subsequent ultrasound images collected along the azimuthal dimension 214 of the transducer array 210 may depict the anatomical structure 304 (and specifically, the specific point 306 on the anatomical structure 304) closer to the horizontal center of such ultrasound images (i.e., closer to the vertical line 308).

FIG. 4 illustrates another example ultrasound image 400, in accordance with certain embodiments described herein. The ultrasound image 400 depicts the anatomical structure 304 and displays the orientation indicator 330. FIG. 4 further illustrates a specific point 306, a vertical line 308, and a distance 318, further description of which may be found with reference FIG. 3. Consider, as an example, that the ultrasound image 400 was collected along the elevational dimension 212 of the transducer array 210. The distance 318 indicates that the specific point 306 on the anatomical structure 304 is to the right of the vertical line 308 in the ultrasound image 400. The position of the orientation indicator 330 to the left of the ultrasound image 400 and the position of the orientation indicator 216 on the upper side of the subject 200 relative to the transducer array 210 of the ultrasound device indicate that rightwards in the ultrasound image 400 corresponds to downwards relative to the subject 200. Thus, the ultrasound device may be too far downwards on the subject 200 and may need to be moved upwards on the subject 200 such that subsequent ultrasound images collected along the elevational dimension 212 of the transducer array 210 may depict the anatomical structure 304 (and specifically, the specific point 306 on the anatomical structure 304) closer to the horizontal center of the ultrasound images (i.e., closer to the vertical line 308). If the ultrasound device collected the ultrasound image 300 and the ultrasound image 400 one after another, then the ultrasound device may be too far leftwards and downwards relative to the subject 200, and may need to be moved both rightwards and upwards, such that subsequent ultrasound images collected both along the azimuthal dimension 214 and the elevational dimension 212 of the transducer array 210 may depict the anatomical structure 304 (and specifically, the specific point 306 on the anatomical structure 304) closer to the horizontal center of the ultrasound images (i.e., closer to the vertical line 308).

FIG. 5 illustrates an example feedback display 500 for positioning an ultrasound device, in accordance with certain embodiments described herein. The feedback provided by the feedback display 500 may be considered of an implicit type. The feedback display 500 includes a marker 524, a vertical line 522, and a horizontal line 520. The vertical line 522 is positioned halfway along the horizontal line 520 and the horizontal line 520 is positioned halfway along the vertical line 522. The position of the marker 524 relative to the vertical line 522 may indicate the direction and magnitude of how far off the ultrasound device is on the left-right dimension of the subject 200, and the position of the marker 524 relative to the horizontal line 520 may indicate the direction and magnitude of how far off the ultrasound device is on the up-down dimension of the subject 200. In particular, the marker 524 indicates how far the ultrasound device is from a location where, if ultrasound images were collected at this location both along the azimuthal dimension 214 and the elevational dimension 212 of the transducer array 210, the anatomical structure 304 (and specifically, the specific point 306 of the anatomical structure 304) would be horizontally centered in both types of ultrasound images.

Consider the ultrasound image 300 of FIG. 3. As described above, if the ultrasound image 300 was collected along the azimuthal dimension 214 of the transducer array 210 of the ultrasound device, and the transducer array 210 is positioned on the subject 200 as displayed in FIG. 2, then the ultrasound device may be too far leftwards relative to the subject 200. Accordingly, the marker 524 is displayed to the left of the vertical line 522, indicating the ultrasound device is too far leftwards relative to the subject 200. Furthermore, the horizontal distance 528 (which may or may not be displayed as part of the feedback display 500) of the marker 524 from the vertical line 522 may be proportional to the distance 318 of the specific point 306 on the anatomical structure 304 from the vertical line 308 in the ultrasound image 300. As the ultrasound device is moved rightwards relative to the subject 200, and the anatomical structure 304 (in particular, the specific point 306 on the anatomical structure 304) moves closer to the horizontal center of ultrasound images collected along the azimuthal dimension 214 of the transducer array 210, the marker 524 may be displayed closer to the vertical line 522.

Consider the ultrasound image 400 of FIG. 4. As described above, if the ultrasound image 400 was collected along the elevational dimension 212 of the transducer array 210 of the ultrasound device, and the transducer array 210 is positioned on the subject 200 as displayed in FIG. 2, then the ultrasound device may be too far downwards on the subject 200. Accordingly, the marker 524 is displayed below the horizontal line 520, indicating the ultrasound device is too far downwards relative to the subject 200. Furthermore, the vertical distance 526 (which may or may not be displayed as part of the feedback display 500) of the marker 524 from the horizontal line 520 may be proportional to the distance 318 of the specific point 306 on the anatomical structure 304 from the vertical line 308 in the ultrasound image 400. As the ultrasound device is moved upwards relative to the subject 200, and the anatomical structure 304 (in particular, the specific point 306 on the anatomical structure 304) moves closer to the horizontal center of ultrasound images collected along the elevational dimension 212 of the transducer array 210, the marker 524 may be displayed closer to the horizontal line 520. A user may move the ultrasound device based on the location of the marker 524 relative to the horizontal line 520 and the vertical line 522 until the marker 524 is located at the intersection of the horizontal line 520 and the vertical line 522 (or within a threshold distance thereof). At this location of the ultrasound device, in both ultrasound images collected along the azimuthal dimension 214 and the elevational dimension 212 of the transducer array 210, the anatomical structure 304 (in particular, the specific point 306 on the anatomical structure 304) may be horizontally centered. It should be appreciated that while this description focuses on horizontal centering, the same methods may be used for vertical centering. For example, the distances calculated from the specific point 306 on the anatomical structure 304 to the center portion of ultrasound images may be measured to a horizontal line positioned halfway along the vertical dimension of the ultrasound images. It should be appreciated that the feedback display 500 may not include any explicit instruction for how to move the ultrasound device.

FIG. 6A illustrates another example feedback display 600A for positioning an ultrasound device, in accordance with certain embodiments described herein. The feedback provided by the feedback display 600A may be considered of an explicit type. The feedback display 600A includes an image of a subject 630A and an arrow 632A. The horizontal component 636A of the arrow 632A (which may or may not be displayed as part of the feedback display 600A) may indicate the direction and magnitude that the ultrasound device may need to be moved relative to the left-right dimension of the subject 200, and the vertical component 634A of the arrow 632A position of the marker 524 relative to the horizontal line 520 may indicate the direction and magnitude that the ultrasound device may need to be moved relative to the up-down dimension of the subject. In particular, the arrow 632A indicates how the ultrasound device may need to be moved such that in ultrasound images collected both along the azimuthal dimension 214 and the elevational dimension 212 of the transducer array 210, the anatomical structure 304 (and specifically, the specific point 306 on the anatomical structure 304) may be horizontally centered in the ultrasound images (i.e., on the vertical line 308).

Consider the ultrasound image 300 of FIG. 3. As described above, if the ultrasound image 300 was collected along the azimuthal dimension 214 of the transducer array 210 of the ultrasound device, and the transducer array 210 is positioned on the subject 200 as displayed in FIG. 2, then the ultrasound device may need to be moved rightwards relative to the subject 200. Accordingly, the arrow 632A is displayed pointing to the right relative to the image of the subject 630A, indicating the ultrasound device should be moved rightwards relative to the subject 200. Furthermore, the horizontal component 636A (which may or may not be displayed as part of the feedback display 500) of the arrow 632A may be proportional to the distance 318 of the specific point 306 on the anatomical structure 304 from the vertical line 308 in the ultrasound image 300. As the ultrasound device is moved rightwards relative to the subject 200, and the anatomical structure 304 (in particular, the specific point 306 on the anatomical structure 304) moves closer to the horizontal center of ultrasound images collected along the azimuthal dimension 214 of the transducer array 210, the horizontal component 636A of the arrow 632A may shrink.

Consider the ultrasound image 400 of FIG. 4. As described above, if the ultrasound image 400 was collected along the elevational dimension 212 of the transducer array 210 of the ultrasound device, and the transducer array 210 is positioned on the subject 200 as displayed in FIG. 2, then the ultrasound device may need to be moved upwards relative to the subject 200. Accordingly, the arrow 632A is displayed pointing upwards relative to the image of the subject 630A, indicating the ultrasound device should be moved upwards relative to the subject 200. Furthermore, the vertical component 634A (which may or may not be displayed as part of the feedback display 600A) of the arrow 632A may be proportional to the distance 318 of the specific point 306 on the anatomical structure 304 from the vertical line 308 in the ultrasound image 400. As the ultrasound device is moved upwards relative to the subject 200, and the anatomical structure 304 (in particular, the specific point 306 on the anatomical structure 304) moves closer to the horizontal center of ultrasound images collected along the elevational dimension 212 of the transducer array 210, the vertical component 634A of the arrow 632A may shrink. A user may move the ultrasound device based on the arrow 632A until the vertical component 634A and the horizontal component 636A of the arrow 632A are with a threshold of zero. At this location of the ultrasound device, in both ultrasound images collected along the azimuthal dimension 214 and the elevational dimension 212 of the transducer array, the anatomical structure 304 (in particular, the specific point 306 on the anatomical structure 304) may be horizontally centered.

FIG. 6B illustrates another example feedback display 600B for positioning an ultrasound device, in accordance with certain embodiments described herein. The feedback provided by the feedback display 600B may be considered of an implicit type. The feedback display 600B simultaneously displays the ultrasound image 300 and the ultrasound image 400, which may be the most recently collected ultrasound images collected along the azimuthal dimension 214 and elevational dimension 212, respectively, of the transducer array 210 of the ultrasound device, and may be updated in real-time as new ultrasound images 300 and 400 are collected. Each of the ultrasound images 300 and 400 depicts the anatomical structure 304 and includes the orientation indicator 330. The feedback display 600B further includes, for each of the ultrasound images 300 and 400, a marker 606B located at the specific point 306 (not shown) on the anatomical structure 304, a vertical line 608B, and a distance 618B. Each of the vertical lines 608B is positioned halfway across the horizontal dimension of the corresponding ultrasound image 300 or 400. Each of the distances 618B is the horizontal distance from the corresponding marker 606B to the corresponding vertical line 608B.

As described above, if the ultrasound image 300 was collected along the azimuthal dimension 214 of the transducer array 210 of the ultrasound device, and the transducer array 210 is positioned on the subject 200 as displayed in FIG. 2, then the ultrasound device may be too far leftwards relative to the subject 200. Furthermore, as described above, if the ultrasound image 400 was collected along the elevational dimension 212 of the transducer array 210 of the ultrasound device, and the transducer array 210 is positioned on the subject 200 as displayed in FIG. 2, then the ultrasound device may be too far downwards on the subject 200. As the ultrasound device is moved rightwards relative to the subject 200, and the anatomical structure 304 (in particular, the specific point 306 on the anatomical structure 304) moves closer to the horizontal center of ultrasound images 300 collected along the azimuthal dimension 214 of the transducer array 210, the marker 606B on the ultrasound image 300 may be displayed closer to the vertical line 608B on the ultrasound image 300, and the distance 618B on the ultrasound image 300 may become smaller. Furthermore. as the ultrasound device is moved upwards relative to the subject 200, and the anatomical structure 304 (in particular, the specific point 306 on the anatomical structure 304) moves closer to the horizontal center of ultrasound images 400 collected along the elevational dimension 212 of the transducer array 210, the marker 606B on the ultrasound image 400 may be displayed closer to the vertical line 608B on the ultrasound image 400, and the distance 618B on the ultrasound image 400 may become smaller. A user may move the ultrasound device based on the location of the markers 606B relative to the horizontal lines 608B until each of the markers 606B is located on or within a threshold distance of the horizontal lines 608B of the respective ultrasound images 300 or 400. At this location of the ultrasound device, in both ultrasound images collected along the azimuthal dimension 214 and the elevational dimension 212 of the transducer array 210, the anatomical structure 304 (in particular, the specific point 306 on the anatomical structure 304) may be horizontally centered. It should be appreciated that while this description focuses on horizontal centering, the same methods may be used for vertical centering. It should be appreciated that the feedback display 600B may not include any explicit instruction for how to move the ultrasound device. It should also be appreciated that in some embodiments, the distances 618B and/or the marker 606B may not be displayed.

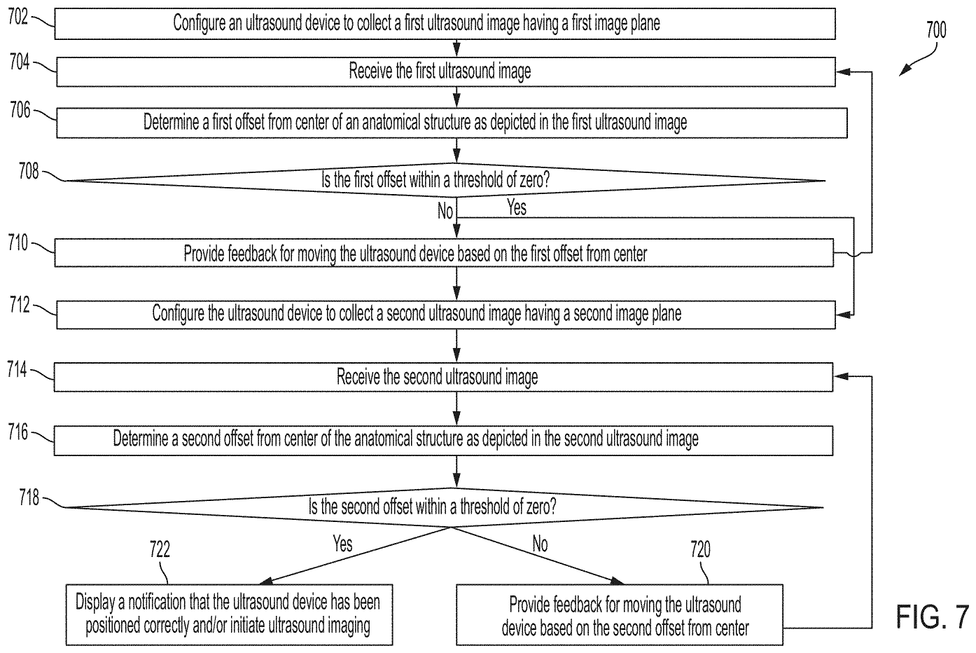

FIG. 7 illustrates another process 700 for providing feedback to a user for positioning an ultrasound device, in accordance with certain embodiments described herein. The process 700 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself (of which the processing device may be a part) may perform the process 700. The process 700 is directed to instructing a user to center an anatomical structure with respect to the transducer array of an ultrasound device.

In act 702, the processing device configures an ultrasound device to collect a first ultrasound image having a first image plane. In some embodiments, the first image plane may be along the azimuthal dimension of the ultrasound device's transducer array. In some embodiments, the first image plane may be along the elevational dimension of the ultrasound device's transducer array. The processing device may configure the ultrasound device and/or may configure itself to use beamforming to focus collection of the first ultrasound image along the first image plane. The process 100 proceeds from act 102 to act 104.

In act 704, the processing device receives the first ultrasound image. Further description of receiving ultrasound images may be found with reference to act 104. The process 700 proceeds from act 704 to act 706.

In act 706, the processing device determines a first offset from center of an anatomical structure as depicted in the first ultrasound image. Further description of determining offsets from center based on ultrasound images may be found with reference to act 106. The process 700 proceeds from act 706 to act 708.

In act 708, the processing device determines if the first offset is within a threshold of zero. Further description of determining if offsets are within a threshold of zero may be found with reference to act 112. If the processing device determines that the first offset is not within a threshold of zero, the process 700 proceeds to act 710. If the processing device determines that the first offset is within a threshold of zero, the process 700 proceeds to act 712.

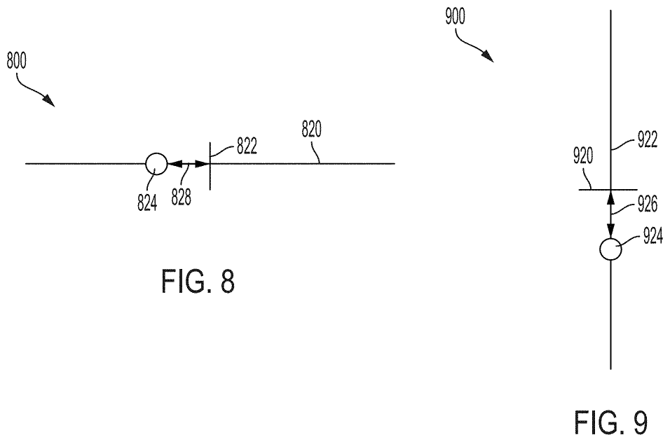

In act 710, the processing device provides feedback for positioning the ultrasound device based on the first offset from center. In some embodiments, the feedback may be to move the ultrasound device to minimize the first offset from center. For example, consider an example in which the first offset from center quantifies a distance on an ultrasound image from a specific point on anatomical structure to a vertical line on the ultrasound image positioned halfway across the horizontal dimension of the ultrasound image. The feedback may be to move the ultrasound device such that subsequent ultrasound images having the first image plane depict the specific point on the anatomical structure closer to the vertical line. In some embodiments, the feedback may be of an implicit type, in which the feedback may indicate the current position of the ultrasound device, and the user may determine based on the current position of the ultrasound device how to move the ultrasound device. For example, the feedback may indicate that the ultrasound device is too far to the left relative to the subject. In some embodiments, the feedback may be of an explicit type, in which the feedback may explicitly instruct the user how to move the ultrasound device. For example, the instruction may indicate that the ultrasound device should be moved rightwards relative to the subject. Further description of such feedback may be found with reference to FIGS. 8-11C. The process 700 proceeds from act 710 to act 704, in which the processing device receives another ultrasound image having the first image plane. In some embodiments, if the ultrasound device is no longer configured to collect ultrasound images having the first image plane, the process 700 may proceed to act 702 instead.

In act 712, the processing device configures the ultrasound device to collect a second ultrasound image having a second image plane. In some embodiments, the second image plane may be orthogonal to the first image plane (although in other embodiments, the first and second image planes may not be orthogonal to each other). In some embodiments, if the first image plane is along the azimuthal dimension of the ultrasound device's transducer array, the second image plane may be along the elevational dimension of the ultrasound device's transducer array. In some embodiments, if the first image plane is along the elevational dimension of the ultrasound device's transducer array, the second image plane may be along the azimuthal dimension of the ultrasound device's transducer array. The processing device may configure the ultrasound device and/or may configure itself to use beamforming to focus collection of the second ultrasound image along the second image plane. The process 700 proceeds from act 712 to act 714.

In act 714, the processing device receives the second ultrasound image. Further description of receiving ultrasound images may be found with reference to act 104. The process 700 proceeds from act 714 to act 716.

In act 716, the processing device determines a second offset from center of the anatomical structure as depicted in the second ultrasound image. Further description of determining offsets from center based on ultrasound images may be found with reference to act 106. The process 700 proceeds from act 716 to act 718.

In act 718, the processing device determines if the second offset is within a threshold of zero. Further description of determining if offsets are within a threshold of zero may be found with reference to act 112. If the processing device determines that the second offset is not within a threshold of zero, the process 700 proceeds from act 718 to act 720. If the processing device determines that the second offset is within a threshold of zero, the process 700 proceeds from act 718 to act 722. In act 722, the processing device displays a notification that the ultrasound device has been positioned correctly and/or initiates (e.g., automatically) ultrasound imaging (e.g., an ultrasound imaging sweep). The process 700 may then terminate. In some embodiments, the processing device may perform another action based on determining, in act 718, that the second offset is both within a threshold of zero. Thus, in some embodiments, act 722 may be omitted.

In act 720, the processing device provides feedback for positioning the ultrasound device based on the second offset from center. Further description of providing feedback for positioning the ultrasound device based on offsets from center may be found with reference to act 708. The process 700 proceeds from act 720 to act 714, in which the processing device receives another ultrasound image having the second image plane. In some embodiments, if the ultrasound device is no longer configured to collect ultrasound images having the second image plane, the process 700 may proceed to act 712 instead.

In some embodiments, act 702 may omitted, for example, if the ultrasound device has been already configured to collect ultrasound images having the first image plane. In some embodiments, acts 708 and 718 may be omitted. In such embodiments, if the first or second offsets are both within a threshold of zero, the feedback provided at acts 710 and 720, respectively, may be to not move the ultrasound device.

In some embodiments, prior to act 712, the processing device may provide an instruction not to move the ultrasound device in a direction that may undo the centering in the first image plane. For example, if the first image plane is along the left-right dimension of the subject, then prior to act 712, the processing device may provide an instruction not to move the ultrasound device along the left-right dimension of the subject.

One difference between the process 700 and the process 100 should be appreciated. In the process 100, the processing device may provide feedback for simultaneously ensuring that the anatomical structure is centered in ultrasound images having two image planes. Accordingly, the processing device configures the ultrasound device to alternate collection of ultrasound images having two image planes. In the process 700, the processing device may provide feedback for ensuring that the anatomical structure is centered in ultrasound images having one image plane, and then provide feedback for ensuring that the anatomical structure is centered in ultrasound image having another image plane. Accordingly, the processing device configures the ultrasound device to collect ultrasound images having a first image plane and then configures the ultrasound device to collect ultrasound images having a second image plane.

FIG. 8 illustrates another example feedback display 800 for positioning an ultrasound device, in accordance with certain embodiments described herein. The feedback provided by the feedback display 800 may be considered of an implicit type. The feedback display 800 includes a marker 824, a horizontal line 820, and a vertical line 822 positioned halfway along the horizontal line 820. The position of the marker 824 relative to the vertical line 822 may indicate the direction and magnitude of how far off the ultrasound device is on the left-right dimension of the subject 200 from a location where in ultrasound images collected either along the azimuthal dimension 214 or the elevational dimension 212 of the transducer array 210, the anatomical structure 304 (and specifically, the specific point 306 on the anatomical structure 304) may be horizontally centered in the ultrasound images (i.e., on the vertical line 308).

Consider the ultrasound image 300 of FIG. 3. As described above, if the ultrasound image 300 was collected along the azimuthal dimension 214 of the transducer array 210 of the ultrasound device, and the transducer array 210 is positioned on the subject 200 as displayed in FIG. 2, then the ultrasound device may be too far leftwards relative to the subject 200. Accordingly, the marker 824 is displayed to the left of the vertical line 822, indicating the ultrasound device is too far leftwards relative to the subject 200. Furthermore, the horizontal distance 828 (which may or may not be displayed as part of the feedback display 800) of the marker 824 from the vertical line 822 may be proportional to the distance 318 of the specific point 306 on the anatomical structure 304 from the vertical line 308 in the ultrasound image 300. As the ultrasound device is moved rightwards relative to the subject 200, and the anatomical structure 304 (in particular, the specific point 306 on the anatomical structure 304) moves closer to the horizontal center of ultrasound images collected along the azimuthal dimension 214 of the transducer array 210, the marker 824 may be displayed closer to the vertical line 822. When the marker 824 is within a threshold distance of the vertical line 822, the ultrasound device may be in a location where in ultrasound images collected along the azimuthal dimension 214 of the transducer array 210, the anatomical structure (in particular, the specific point 306 on the anatomical structure 304) is horizontally centered, and the feedback display 900 may then be displayed. It should be appreciated that the feedback display 800 may not include any explicit instruction for how to move the ultrasound device.

FIG. 9 illustrates another example feedback display 900 for positioning an ultrasound device, in accordance with certain embodiments described herein. The feedback provided by the feedback display 900 may be considered of an implicit type. The feedback display 900 includes a marker 924, a vertical line 922, and a horizontal line 920 positioned halfway along the vertical line 922. The position of the marker 924 relative to the horizontal line 920 may indicate the direction and magnitude of how far off the ultrasound device is on the up-down dimension of the subject 200 from a location where in ultrasound images collected either along the azimuthal dimension 214 or the elevational dimension 212 of the transducer array 210, the anatomical structure 304 (and specifically, the specific point 306 on the anatomical structure 304) is horizontally centered in the ultrasound images (i.e., on the vertical line 308).

Consider the ultrasound image 400 of FIG. 4. As described above, if the ultrasound image 400 was collected along the elevational dimension 212 of the transducer array 210 of the ultrasound device, and the transducer array 210 is positioned on the subject 200 as displayed in FIG. 2, then the ultrasound device may be too far downwards on the subject. Accordingly, the marker 924 is displayed below the horizontal line 920, indicating the ultrasound device is too far downwards relative to the subject 200. Furthermore, the vertical distance 926 (which may or may not be displayed as part of the feedback display 900) of the marker 924 from the horizontal line 920 may be proportional to the distance 318 of the specific point 306 on the anatomical structure 304 from the vertical line 308 in the ultrasound image 400. As the ultrasound device is moved upwards relative to the subject 200, and the anatomical structure 304 (in particular, the specific point 306 on the anatomical structure 304) moves closer to the horizontal center of ultrasound images collected along the elevational dimension 212 of the transducer array 210, the marker 928 may be displayed closer to the horizontal line 920. When the marker 924 is within a threshold distance of the horizontal line 920 (and the marker 824 has already been brought within a threshold distance of the vertical line 822), the ultrasound device may be in a location where in both ultrasound images collected along the azimuthal dimension 214 and the elevational dimension 212 of the transducer array, the anatomical structure 304 (in particular, the specific point 306 on the anatomical structure 304) is horizontally centered. It should be appreciated that the feedback display 900 may not include any explicit instruction for how to move the ultrasound device.

It should thus be appreciated from the above description that the feedback display 800 first may provide feedback for ensuring that the anatomical structure 304 is centered in ultrasound images collected along the azimuthal dimension 214 of the transducer array 210, and then the feedback display 900 may provide feedback for ensuring that the anatomical structure 304 is centered in ultrasound images collected along the elevational dimension 212 of the transducer array 210. In some embodiments, however, feedback may first ensure that the anatomical structure 304 is centered in ultrasound images collected along the elevational dimension 212 of the transducer array 210, and then feedback may ensure that the anatomical structure 304 is centered in ultrasound images collected along the azimuthal dimension 214 of the transducer array 210.

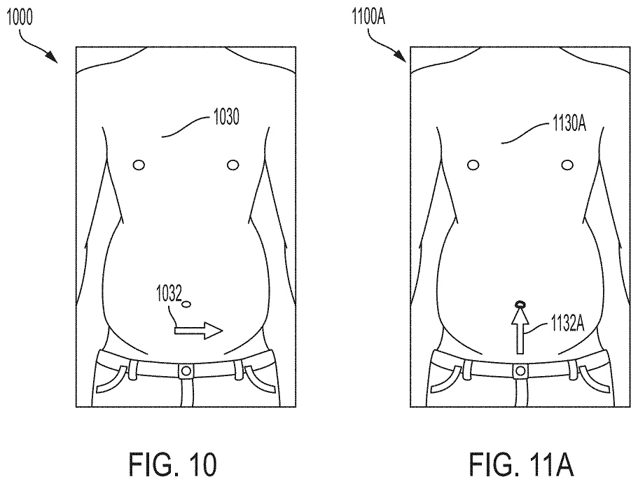

FIG. 10 illustrates another example feedback display 1000 for positioning an ultrasound device, in accordance with certain embodiments described herein. The feedback provided by the feedback display 1000 may be considered of an explicit type. The feedback display 1000 includes an image of a subject 1030 and an arrow 1032. The arrow 1032 may indicate the direction and magnitude that the ultrasound device may need to be moved relative to the left-right dimension of the subject 200, such that in ultrasound images collected either along the azimuthal dimension 214 or the elevational dimension 212 of the transducer array 210, the anatomical structure 304 (and specifically, the specific point 306 on the anatomical structure 304) may be horizontally centered in the ultrasound images (i.e., on the vertical line 308).

Consider the ultrasound image 300. As described above, if the ultrasound image 300 was collected along the azimuthal dimension 214 of the transducer array 210 of the ultrasound device, and the transducer array 210 is positioned on the subject 200 as displayed in FIG. 2, then the ultrasound device may need to be moved rightwards relative to the subject 200. Accordingly, the arrow 1032 is displayed pointing to the right relative to the image of the subject 1030, indicating the ultrasound device should be moved rightwards relative to the subject 200. Furthermore, the length of the arrow 1032 may be proportional to the distance 318 of the specific point 306 on the anatomical structure 304 from the vertical line 308 in the ultrasound image 300. As the ultrasound device is moved rightwards relative to the subject 200, and the anatomical structure 304 (in particular, the specific point 306 on the anatomical structure 304) moves closer to the horizontal center of ultrasound images collected along the azimuthal dimension 214 of the transducer array 210, the length of the arrow 1032 may shrink. When the length of the arrow 1032 is within a threshold of zero, the ultrasound device may be in a location where in ultrasound images collected along the azimuthal dimension 214 of the transducer array 210, the anatomical structure 304 (in particular, the specific point 306 on the anatomical structure 304) is horizontally centered, and the feedback display 1100A may then be displayed.

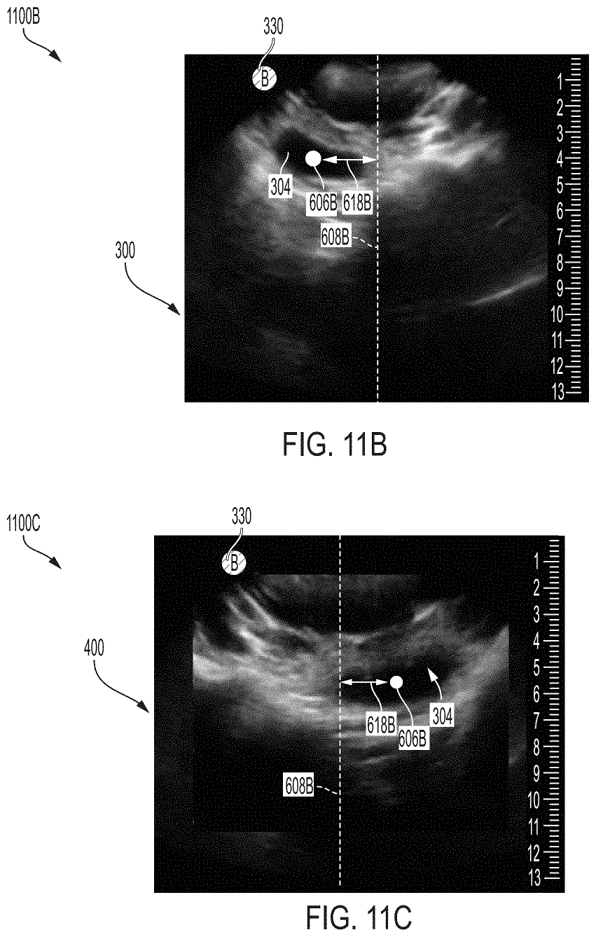

FIG. 11A illustrates another example feedback display 1100A for positioning an ultrasound device, in accordance with certain embodiments described herein. The feedback provided by the feedback display 1100A may be considered of an explicit type. The feedback display 1100A includes an image of a subject 1130A and an arrow 1132A. The arrow 1132A may indicate the direction and magnitude that the ultrasound device may need to be moved relative to the up-down dimension of the subject 200, such that in ultrasound images collected either along the azimuthal dimension 214 or the elevational dimension 212 of the transducer array 210, the anatomical structure 304 (and specifically, the specific point 306 on the anatomical structure 304) is horizontally centered in the ultrasound images (i.e., on the vertical line 308).

Consider the ultrasound image 400. As described above, if the ultrasound image 400 was collected along the elevational dimension 212 of the transducer array 210 of the ultrasound device, and the transducer array 210 is positioned on the subject 200 as displayed in FIG. 2, then the ultrasound device may need to be moved upwards relative to the subject 200. Accordingly, the arrow 1132A is displayed pointing upwards relative to the image of the subject 1130A, indicating the ultrasound device should be moved upwards relative to the subject 200. Furthermore, the length of the arrow 1132A may be proportional to the distance 318 of the specific point 306 on the anatomical structure 304 from the vertical line 308 in the ultrasound image 400. As the ultrasound device is moved upwards relative to the subject 200, and the anatomical structure 304 (in particular, the specific point 306 on the anatomical structure 304) moves closer to the horizontal center of ultrasound images collected along the elevational dimension 212 of the transducer array 210, the vertical component 1134 of the arrow 1132A may shrink. When the length of the arrow 1132A is within a threshold of zero (and the length of the arrow 1032 was previously shrunk to within a threshold zero), the ultrasound device may be in a location where in ultrasound images collected both along the azimuthal dimension 214 and the elevational dimension 212 of the transducer array 210, the anatomical structure 304 (in particular, the specific point 306 on the anatomical structure 304) is horizontally centered.

It should thus be appreciated from the above description that the feedback display 1000 first may provide feedback for ensuring that the anatomical structure 304 is centered in ultrasound images collected along the azimuthal dimension 214 of the transducer array 210, and then the feedback display 1100A may provide feedback for ensuring that the anatomical structure 304 is centered in ultrasound images collected along the elevational dimension 212 of the transducer array 210. In some embodiments, however, feedback may first ensure that the anatomical structure 304 is centered in ultrasound images collected along the elevational dimension 212 of the transducer array 210, and then feedback may ensure that the anatomical structure 304 is centered in ultrasound images collected along the azimuthal dimension 214 of the transducer array 210.

FIG. 11B illustrates another example feedback display 1100B for positioning an ultrasound device, in accordance with certain embodiments described herein. The feedback provided by the feedback display 1100B may be considered of an implicit type. The feedback display 1100B displays the ultrasound image 300, which may be the most recently collected ultrasound image collected along the azimuthal dimension 214 of the transducer array 210 of the ultrasound device, and may be updated in real-time as new ultrasound images 300 are collected. The ultrasound image 300 depicts the anatomical structure 304 and includes the orientation indicator 330. The feedback display 1100B further includes the marker 606B located at the specific point (not shown) 306 on the anatomical structure 304, the vertical line 608B, and the distance 618B.

As described above, if the ultrasound image 300 was collected along the azimuthal dimension 214 of the transducer array 210 of the ultrasound device, and the transducer array 210 is positioned on the subject 200 as displayed in FIG. 2, then the ultrasound device may be too far leftwards relative to the subject 200. As the ultrasound device is moved rightwards relative to the subject 200, and the anatomical structure 304 (in particular, the specific point 306 on the anatomical structure 304) moves closer to the horizontal center of ultrasound images 300 collected along the azimuthal dimension 214 of the transducer array 210, the marker 606B may be displayed closer to the vertical line 608B, and the distance 618B on the ultrasound image 300 may become smaller. A user may move the ultrasound device based on the location of the marker 606B relative to the horizontal line 608B until the marker 606B is located on or within a threshold distance of the horizontal line 608B. At this location of the ultrasound device, in ultrasound images collected along the azimuthal dimension 214 of the transducer array 210, the anatomical structure 304 (in particular, the specific point 306 on the anatomical structure 304) may be horizontally centered, and the feedback display 1100B may then be displayed. It should be appreciated that while this description focuses on horizontal centering, the same methods may be used for vertical centering. It should be appreciated that the feedback display 1100B may not include any explicit instruction for how to move the ultrasound device. It should also be appreciated that in some embodiments, the distances 618B and/or the marker 606B may not be displayed.

FIG. 11C illustrates another example feedback display 1100C for positioning an ultrasound device, in accordance with certain embodiments described herein. The feedback provided by the feedback display 1100C may be considered of an implicit type. The feedback display 1100C displays the ultrasound image 400, which may be the most recently collected ultrasound image collected along the elevational dimension 212 of the transducer array 210 of the ultrasound device, and may be updated in real-time as new ultrasound images 400 are collected. The ultrasound image 400 depicts the anatomical structure 304 and includes the orientation indicator 330. The feedback display 1100C further includes the marker 606B located at the specific point 306 (not shown) on the anatomical structure 304, the vertical line 608B, and the distance 618B.

As described above, if the ultrasound image 400 was collected along the elevational dimension 212 of the transducer array 210 of the ultrasound device, and the transducer array 210 is positioned on the subject 200 as displayed in FIG. 2, then the ultrasound device may be too far downwards on the subject 200. As the ultrasound device is moved upwards relative to the subject 200, and the anatomical structure 304 (in particular, the specific point 306 on the anatomical structure 304) moves closer to the horizontal center of ultrasound images 400 collected along the elevational dimension 212 of the transducer array 210, the marker 606B may be displayed closer to the vertical line 608B, and the distance 618B may become smaller. A user may move the ultrasound device based on the location of the marker 606B relative to the horizontal line 608B until the marker 606B is located on or within a threshold distance of the horizontal line 608B. At this location of the ultrasound device, in ultrasound images collected along the elevational dimension 212 of the transducer array 210, the anatomical structure 304 (in particular, the specific point 306 on the anatomical structure 304) may be horizontally centered. It should be appreciated that while this description focuses on horizontal centering, the same methods may be used for vertical centering. It should be appreciated that the feedback display 1100C may not include any explicit instruction for how to move the ultrasound device. It should also be appreciated that in some embodiments, the distances 618B and/or the marker 606B may not be displayed.

It should thus be appreciated from the above description that the feedback display 1100B first may provide feedback for ensuring that the anatomical structure 304 is centered in ultrasound images collected along the azimuthal dimension 214 of the transducer array 210, and then the feedback display 1100C may provide feedback for ensuring that the anatomical structure 304 is centered in ultrasound images collected along the elevational dimension 212 of the transducer array 210. In some embodiments, however, feedback may first ensure that the anatomical structure 304 is centered in ultrasound images collected along the elevational dimension 212 of the transducer array 210, and then feedback may ensure that the anatomical structure 304 is centered in ultrasound images collected along the azimuthal dimension 214 of the transducer array 210.

It should be appreciated that as referred to herein, positioning the ultrasound device may refer to translating an ultrasound device and/or tilting the ultrasound device. For example, positioning an ultrasound device rightwards may include translating the ultrasound device rightwards to a new position, or tilting the ultrasound device about its current location such that the transducer array of the ultrasound device faces the rightwards direction to a greater degree. It should thus be appreciated that while the feedback display 1000 and 1100A may indicate that the ultrasound device should be translated to a new position, in some embodiments, feedback may indicate that the ultrasound device should be tilted about its current position.

It should be appreciated that while the above description has focused on feedback to move the ultrasound device along the left-right and/or up-down dimensions of the subject 200, this may be due to the particular orientation of the transducer array 210 in FIG. 2. If, for example, the azimuthal dimension 214 and the elevational dimension 212 of the transducer array 210 were not oriented along the left-right and up-down dimensions of the subject 200, then the feedback may be to move the ultrasound device in other directions relative to the subject 200. It should also be appreciated that the transducer array 210 may have various shapes, and therefore the image planes may be of various types.

While the above description has described centering an anatomical structure in ultrasound images have two image planes, in some embodiments, three or more image planes may be used. In such embodiments, feedback (either of the implicit or explicit type) may be provided to center the anatomical structure in ultrasound images having all the different image planes (e.g., to center the anatomical structure in ultrasound images having first, second, and third image planes). For example, two image planes separated by 90 degrees, three image planes separated by 60 degrees, four image planes separated by 45 degrees, or more generally, N image planes separated by 180/N degrees, may be used. Centering of the anatomical structure may be more accurate if more image planes are used, but frame rate may be reduced such that feedback is updated less frequently. Given a certain number of image planes, centering of the anatomical structure may be more accurate if components of the image planes overlap less (e.g., error may be less for two image planes separated by 90 degrees versus two image planes separated by 70 degrees).

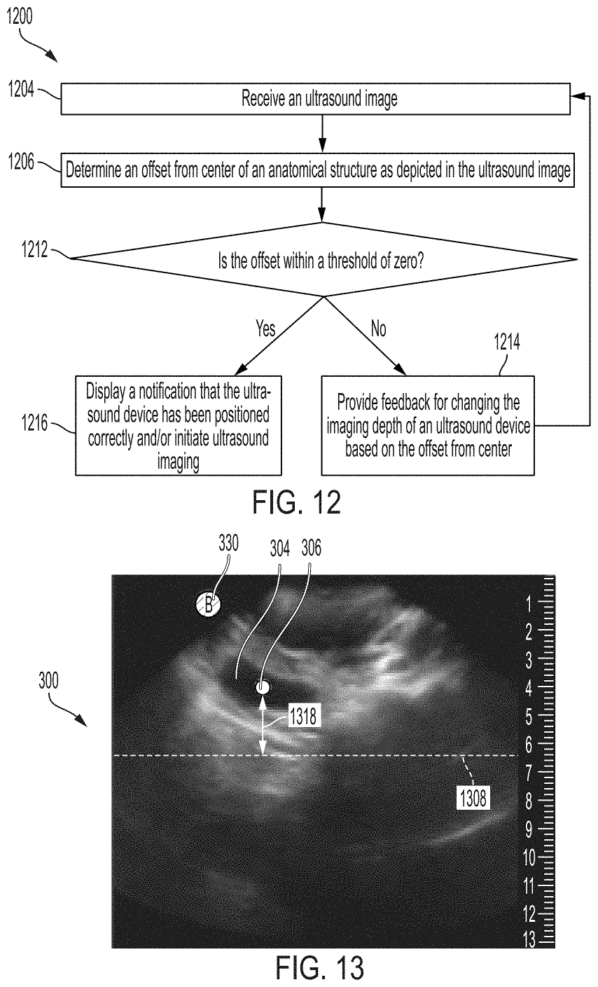

FIG. 12 illustrates another process 1200 for providing feedback to a user for changing the imaging depth of an ultrasound device, in accordance with certain embodiments described herein. The process 1200 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself (of which the processing device may be a part) may perform the process 1200. The process 1200 is directed to instructing a user to center an anatomical structure with respect to the depth dimension of the field of view of an ultrasound device.

In act 1204, the processing device receives an ultrasound image. Further description of receiving ultrasound images may be found with reference to act 104. The process 1200 proceeds from act 1204 to act 1206.

In act 1206, the processing device determines an offset from center of an anatomical structure as depicted in the ultrasound image. Further description of determining offsets from center based on ultrasound images may be found with reference to act 106. As described with reference to act 106, the offset from center may correspond to a distance of the anatomical structure (e.g., a bladder) depicted in the ultrasound image from a center portion of the ultrasound image. In some embodiments, the center portion of the ultrasound image may be a horizontal line positioned halfway across a vertical dimension of the first ultrasound image, and the processing device may measure the vertical distance from the specific point on the anatomical structure to the horizontal line. This may be the case if the vertical dimension of the ultrasound image is parallel to the depth dimension of the field of view of the ultrasound device. In some embodiments, the center portion of the ultrasound image may be a vertical line positioned halfway across a horizontal dimension of the first ultrasound image, and the processing device may measure the horizontal distance from the specific point on the anatomical structure to the vertical line. This may be the case if the horizontal dimension of the ultrasound image is parallel to the depth dimension of the field of view of the ultrasound device. The process 1200 proceeds from act 1206 to act 1208.

In act 1212, the processing device determines if the offset is within a threshold of zero. Further description of determining if offsets are within a threshold of zero may be found with reference to act 112. If the processing device determines that the offset is not within a threshold of zero, the process 1200 proceeds to act 1214. If the processing device determines that the offset is within a threshold of zero, the process 1200 proceeds to act 1216.

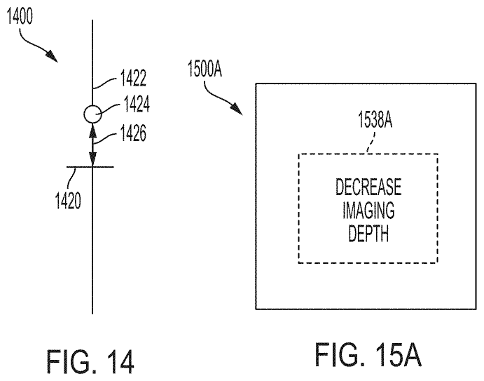

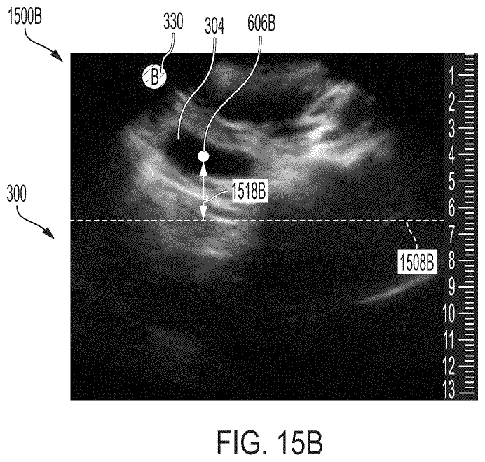

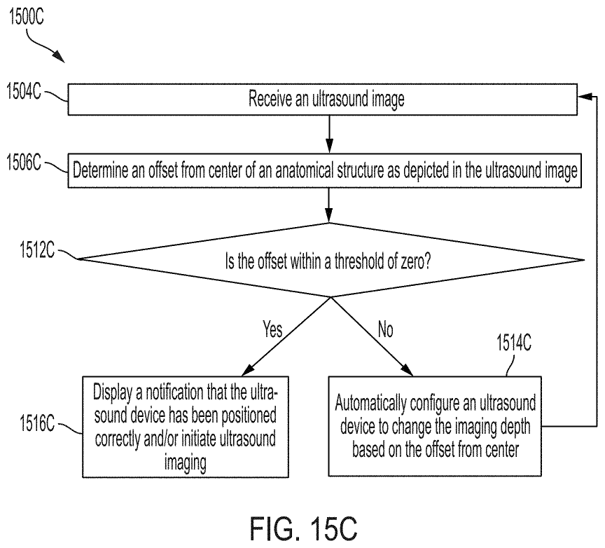

In act 1214, the processing device provides feedback for changing the imaging depth of the ultrasound device based on the offset from center. In some embodiments, the feedback may be to change the imaging depth to minimize the offset from center. For example, consider an example in which the offset from center quantifies a distance on an ultrasound image from a specific point on an anatomical structure to a horizontal line on the ultrasound image positioned halfway across the vertical dimension of the ultrasound image. The feedback may be to change the imaging depth such that subsequent ultrasound images depict the anatomical structure closer to the horizontal line. In some embodiments, the feedback may be of an implicit type, in which the feedback may indicate the current position of a specific point on an anatomical structure relative to a horizontal line on the ultrasound image, and the user may determine based on the current position how to change the imaging depth. For example, the feedback may indicate that the specific point on the anatomical structure is above the horizontal line on the ultrasound image, and thus the imaging depth should be decreased. In such embodiments, the feedback may not include an explicit instruction to change the imaging depth. In some embodiments, the feedback may be of an explicit type, in which the feedback may explicitly instruct the user how to change the imaging depth. For example, the feedback may indicate that the imaging depth should be decreased. Further description of such feedback may be found with reference to FIGS. 14-15B, described in further detail hereinafter.

In act 1216 the processing device displays a notification that the ultrasound device has been positioned correctly and/or initiates (e.g., automatically) ultrasound imaging (e.g., an ultrasound imaging sweep). The process 1200 may then terminate. In some embodiments, the processing device may perform another action based on determining, in act 1212, that the offset is within a threshold of zero. Thus, in some embodiments, act 1216 may be omitted.

FIG. 13 illustrates the example ultrasound image 300, in accordance with certain embodiments described herein. The ultrasound image 300 depicts the anatomical structure 304 (in the example of FIG. 13, a bladder), and includes the orientation indicator 330. The orientation indicator 330 is positioned on the left of the ultrasound image 300, but may be positioned on the right of the ultrasound images. FIG. 13 further illustrates the specific point 306 on the anatomical structure 304, a horizontal line 1308, and a distance 1318. The horizontal line 1308 is positioned halfway across the vertical dimension of the ultrasound image 300. The distance 1318 is the vertical distance from the specific point 306 to the horizontal line 1308. In the example of FIG. 13, the imaging depth is 13 cm, the horizontal line 1308 is located at 6.5 cm, and the specific point 306 is located at a depth of approximately 4 cm. Thus, the distance 1318 is approximately 2.5 cm, and the specific point 306 is shallower than the depth of the horizontal line 1308. The imaging depth may thus be too large. To reduce the distance 1318 to within a threshold zero, the imaging depth may need to be changed such that the horizontal line 1308 is located at the same depth as the specific point 306. Thus, the imaging depth may need to be changed by approximately 5 cm, to approximately 8 cm, such that the horizontal line 1308 is positioned at approximately 4 cm. It should be appreciated that the distance 1318 may be proportional to (e.g., half as much as) the required change in imaging depth. It should also be appreciated that ultrasound images along other image planes (i.e., besides the image plane of the ultrasound image 300) may be used.

FIG. 14 illustrates another example feedback display 1400 for positioning an ultrasound device, in accordance with certain embodiments described herein. The feedback provided by the feedback display 1400 may be considered of an implicit type. The feedback display 1400 includes a marker 1424, a vertical line 1422, and a horizontal line 1420 positioned halfway along the vertical line 1422. The position of the marker 1424 relative to the horizontal line 1420 may indicate the direction and magnitude of how much the imaging depth of ultrasound images must change such that the anatomical structure 304 (and specifically, the specific point 306 on the anatomical structure 304) is vertically centered in the ultrasound images (i.e., on the vertical line 308).

Consider the ultrasound image 300 of FIG. 13. As described above, the imaging depth may be too large. Accordingly, the marker 1424 is displayed above the horizontal line 1420, indicating the imaging depth is too large. Furthermore, the vertical distance 1426 (which may or may not be displayed as part of the feedback display 1400) of the marker 1424 from the horizontal line 1420 may be proportional to the distance 1318 of the specific point 306 on the anatomical structure 304 from the horizontal line 1308 in the ultrasound image 1300. As the imaging depth is decreased, and the anatomical structure 304 (in particular, the specific point 306 on the anatomical structure 304) moves closer to the vertical center of collected ultrasound images, the marker 1424 may be displayed closer to the horizontal line 1420. When the marker 1424 is within a threshold distance of the horizontal line 1420, the imaging depth may be set such that in collected ultrasound images, the anatomical structure 304 (in particular, the specific point 306 on the anatomical structure 304) is vertically centered. In some embodiments, an indication may be provided on the feedback display 1400 and/or the feedback display 900 indicating that for the feedback display 1400, the imaging depth should be changed, while for the feedback display 900, the ultrasound device should be moved.

FIG. 15A illustrates another example feedback display 1500A for positioning an ultrasound device, in accordance with certain embodiments described herein. The feedback provided by the feedback display 1500A may be considered of an explicit type. The feedback display 1500A includes text 1538A indicating that the imaging depth should be decreased (although it may alternatively indicate that the imaging depth should be increased). Consider the ultrasound image 300 of FIG. 13. As described above, the imaging depth may be too large. The feedback display 1500A may display the text 1538A until the imaging depth is decreased such that in collected ultrasound images, the anatomical structure 304 (in particular, the specific point 306 on the anatomical structure 304) is vertically centered.

FIG. 15B illustrates another example feedback display 1500B for positioning an ultrasound device, in accordance with certain embodiments described herein. The feedback provided by the feedback display 1500B may be considered of an implicit type. The feedback display 1500B displays the ultrasound image 300, which may be the most recently collected ultrasound image, and may be updated in real-time as new ultrasound images 300 are collected. It should also be appreciated that ultrasound images along other image planes (i.e., besides the image plane of the ultrasound image 300) may be used. The ultrasound image 300 depicts the anatomical structure 304 and includes the orientation indicator 330. The feedback display 1500B further includes the marker 606B located at the specific point 306 (not shown) on the anatomical structure 304, a horizontal line 1508B, and a distance 1518B. The horizontal line 608B is positioned halfway across the vertical dimension of the ultrasound image 300. The distances 1518B is the vertical distance from the marker 606B to the horizontal line 1508B. As described above, the imaging depth may be too large in the ultrasound image 300. As the imaging depth is decreased, and the anatomical structure 304 (in particular, the specific point 306 on the anatomical structure 304) moves closer to the vertical center of collected ultrasound images, the horizontal line 1508B may move closer to the marker 606B. When the marker horizontal line 1508B is within a threshold distance of the marker 606B, the imaging depth may be set such that in collected ultrasound images, the anatomical structure 304 (in particular, the specific point 306 on the anatomical structure 304) is vertically centered.

FIG. 15C illustrates a process 1500C for automatically changing the imaging depth of an ultrasound device, in accordance with certain embodiments described herein. The process 1500C is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself (of which the processing device may be a part) may perform the process 1500C. The process 1500C is directed to automatically centering an anatomical structure with respect to the depth dimension of the field of view of an ultrasound device.

Acts 1504C, 1506C, 1512C, and 1516C are the same as acts 1204, 1206, 1212, and 1216, respectively, of the process 1200. In act 1514C, the processing device automatically configures the ultrasound device to change the imaging depth based on the offset from center. In some embodiments, the processing device may transmit commands to the ultrasound device to change the imaging depth. In some embodiments, the processing device may calculate the imaging depth value that will minimize the offset from center, and configure the ultrasound device to change the imaging depth to that value. For example, if the imaging depth is 13 cm and the specific point 306 of the anatomical region 304 is located at a depth of 4 cm, then the processing device may calculate that the imaging depth should be changed to 8 cm. In some embodiments, the processing device may configure the ultrasound device to incrementally increase or decrease the imaging depth until the processing device determines that at a particular imaging depth, the offset from center has been minimized. It should be appreciated that while the process 1200 describes providing a feedback to a user such that the user will change the imaging depth, the process 1500C describes automatically changing the imaging depth, without providing feedback to the user or the user changing the imaging depth himself or herself.

In some embodiments, the processing device may provide feedback for centering the anatomical structure with respect to the transducer array of the ultrasound device (e.g., perform the process 100 or the process 700) and then provide feedback for centering the anatomical structure with respect to the depth dimension of the field of view of the ultrasound device (e.g., perform the process 1200) or automatically center the anatomical structure with respect to the depth dimension of the field of view of the ultrasound device (e.g., perform the process 1500C). In some embodiments, the processing device may provide feedback for centering the anatomical structure with respect to the depth dimension of the field of view of the ultrasound device (e.g., perform the process 1200) or automatically center the anatomical structure with respect to the depth dimension of the field of view of the ultrasound device (e.g., perform the process 1500C) and then provide feedback for centering the anatomical structure with respect to the transducer array of the ultrasound device (e.g., perform the process 100 or the process 700).

It should be appreciated that the forms of the feedback described herein are not limiting and other forms conveying the same information may be used. For example, other types of direction indicators may be used instead of arrows, other types of markers may be used instead of circles, and other types of means for displaying offsets from center may be used. In some embodiments, rather than displaying graphical feedback, textual feedback may be displayed, or audio feedback may be played. In some embodiments, the feedback displays shown may be shown adjacent to, or overlaid on, a graphical user interface for collecting ultrasound images.

While the above description has used the bladder as an exemplary anatomical structure, the methods and apparatuses described herein may also be applied to guiding collection of ultrasound images of the thyroid, the abdominal aorta, a superficial artery, the brain (e.g., a neonatal brain), the liver, the breast, the kidney, and amniotic fluid. Example applications include venous access identification when imaging a superficial artery; imaging benign hemangiomas in the liver; imaging nodules in the thyroid, imaging cancerous tumors in the liver, breast, kidney, and pancreas to detect changes over time; and amniotic fluid evaluation.

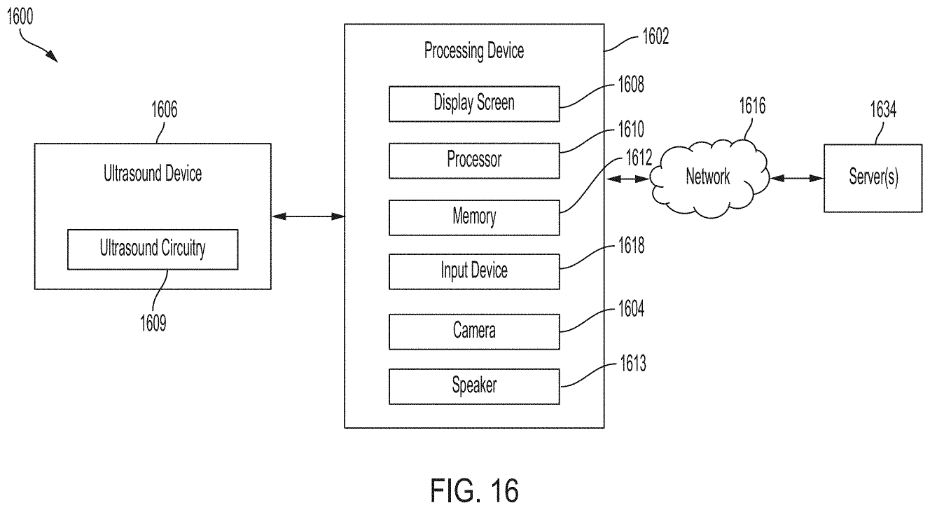

FIG. 16 illustrates a schematic block diagram of an example ultrasound system 1600 upon which various aspects of the technology described herein may be practiced. The ultrasound system 1600 includes an ultrasound device 1606, a processing device 1602, a network 1616, and one or more servers 1634.

The ultrasound device 1606 includes ultrasound circuitry 1609. The processing device 1602 includes a camera 1604, a display screen 1608, a processor 1610, a memory 1612, an input device 1618, and a speaker 1613. The processing device 1602 is in wired (e.g., through a lightning connector or a mini-USB connector) and/or wireless communication (e.g., using BLUETOOTH, ZIGBEE, and/or WiFi wireless protocols) with the ultrasound device 1606. The processing device 1602 is in wireless communication with the one or more servers 1634 over the network 1616. However, the wireless communication with the one or more servers 1634 is optional.

The ultrasound device 1606 may be configured to generate ultrasound data that may be employed to generate an ultrasound image. The ultrasound device 1606 may be constructed in any of a variety of ways. In some embodiments, the ultrasound device 1606 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 1609 may be configured to generate the ultrasound data. The ultrasound circuitry 1609 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 1609 (e.g., transmit circuitry, receive circuitry, control circuitry, power management circuitry, and processing circuitry) to form a monolithic ultrasound device. The ultrasound device 1606 may transmit ultrasound data and/or ultrasound images to the processing device 1602 over a wired (e.g., through a lightning connector or a mini-USB connector) and/or wireless (e.g., using BLUETOOTH, ZIGBEE, and/or WiFi wireless protocols) communication link.

Referring now to the processing device 1602, the processor 1610 may include specially-programmed and/or special-purpose hardware such as an application-specific integrated circuit (ASIC). For example, the processor 1610 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 1602 may be configured to process the ultrasound data received from the ultrasound device 1606 to generate ultrasound images for display on the display screen 1608. The processing may be performed by, for example, the processor 1610. The processor 1610 may also be adapted to control the acquisition of ultrasound data with the ultrasound device 1606. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 1602 may be configured to perform certain of the processes (e.g., the process 10) described herein using the processor 1610 (e.g., one or more computer hardware processors) and one or more articles of manufacture that include non-transitory computer-readable storage media such as the memory 1612. The processor 1610 may control writing data to and reading data from the memory 1612 in any suitable manner. To perform certain of the processes described herein, the processor 1610 may execute one or more processor-executable instructions stored in one or more non-transitory computer-readable storage media (e.g., the memory 1612), which may serve as non-transitory computer-readable storage media storing processor-executable instructions for execution by the processor 1610. The camera 1604 may be configured to detect light (e.g., visible light) to form an image. The camera 1604 may be on the same face of the processing device 1602 as the display screen 1608. The display screen 1608 may be configured to display images and/or videos, and may be, for example, a liquid crystal display (LCD), a plasma display, and/or an organic light emitting diode (OLED) display on the processing device 1602. The input device 1618 may include one or more devices capable of receiving input from a user and transmitting the input to the processor 1610. For example, the input device 1618 may include a keyboard, a mouse, a microphone, touch-enabled sensors on the display screen 1608, and/or a microphone. The display screen 1608, the input device 1618, the camera 1604, and the speaker 1613 may be communicatively coupled to the processor 1610 and/or under the control of the processor 1610.

It should be appreciated that the processing device 1602 may be implemented in any of a variety of ways. For example, the processing device 1602 may be implemented as a handheld device such as a mobile smartphone or a tablet. Thereby, a user of the ultrasound device 1606 may be able to operate the ultrasound device 1606 with one hand and hold the processing device 1602 with another hand. In other examples, the processing device 1602 may be implemented as a portable device that is not a handheld device, such as a laptop. In yet other examples, the processing device 1602 may be implemented as a stationary device such as a desktop computer. The processing device 1602 may be connected to the network 1616 over a wired connection (e.g., via an Ethernet cable) and/or a wireless connection (e.g., over a WiFi network). The processing device 1602 may thereby communicate with (e.g., transmit data to) the one or more servers 1634 over the network 1616. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 and published as U.S. Pat. App. Publication No. 2017-0360397 A1 (and assigned to the assignee of the instant application).

FIG. 16 should be understood to be non-limiting. For example, the ultrasound system 1600 may include fewer or more components than shown and the processing device 1602 and ultrasound device 1606 may include fewer or more components than shown. In some embodiments, the processing device 1602 may be part of the ultrasound device 1606.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various inventive concepts may be embodied as one or more processes, of which an example has been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
a processing device in operative communication with an ultrasound device, the processing device configured to:
configure the ultrasound device to collect one or more first ultrasound images having a first image plane and one or more second ultrasound images having a second image plane; and
provide feedback to a user for positioning the ultrasound device based on the one or more first ultrasound images having the first image plane and/or the one or more second ultrasound images having the second image plane.

2. The apparatus of claim 1, wherein the processing device is configured, when configuring the ultrasound device to collect the one or more first ultrasound images having the first image plane and the one or more second ultrasound images having the second image plane, to configure the ultrasound device to alternate collection of ultrasound images having the first image plane ultrasound images having the second image plane.

3. The apparatus of claim 2, wherein the processing device is configured, when configuring the ultrasound device to alternate collection of ultrasound images having the first image plane ultrasound images having the second image plane, to configure the ultrasound device to alternate the collection at a frame rate within a range of approximately 15-30 Hz.

4. The apparatus of claim 2, wherein the processing device is configured, when configuring the ultrasound device to alternate collection of ultrasound images having the first image plane ultrasound images having the second image plane, to configure the ultrasound device to collect one ultrasound image having the first image plane and one ultrasound image having the second image plane.

5. The apparatus of claim 1, wherein the processing device is configured, when configuring the ultrasound device to collect the one or more first ultrasound images having the first image plane and the one or more second ultrasound images having the second image plane, to configure the ultrasound device to collect the one or more first ultrasound images and subsequently configuring the ultrasound device to collect the one or more second ultrasound images.

6. The apparatus of claim 1, wherein the processing device is configured, when configuring the ultrasound device to collect the one or more first ultrasound images having the first image plane and the one or more second ultrasound images having the second image plane, to configure the ultrasound device and/or itself to use beamforming to collect the one or more first ultrasound images having the first image plane and the one or more second ultrasound images having the second image plane.

7. The apparatus of claim 1, wherein the processing device is configured, when providing the feedback to the user for positioning the ultrasound device based on the one or more first ultrasound images having the first image plane and/or the one or more second ultrasound images having the second image plane, to provide feedback for centering an anatomical structure depicted in the one or more first ultrasound images having the first image plane and the one or more second ultrasound images having the second image plane.

8. The apparatus of claim 7, wherein the anatomical structure comprises a bladder.

9. The apparatus of claim 7, wherein the processing device is further configured to perform an ultrasound imaging sweep subsequent to determining that the anatomical structure depicted in the one or more first ultrasound images having the first image plane and the one or more second ultrasound images having the second image plane is centered.

10. The apparatus of claim 7, wherein the processing device is configured, when providing the feedback for centering the anatomical structure depicted in the one or more first ultrasound images having the first image plane and the one or more second ultrasound images having the second image plane, to provide feedback for simultaneously centering the anatomical structure depicted in the one or more first ultrasound images having the first image plane and the one or more second ultrasound images having the second image plane.

11. The apparatus of claim 10, wherein the processing device is configured, when providing the feedback for simultaneously centering the anatomical structure depicted in the one or more first ultrasound images having the first image plane and the one or more second ultrasound images having the second image plane, to:
receive a first ultrasound image having the first image plane;
determine a first offset from center of the anatomical structure depicted in the first ultrasound image;
receive a second ultrasound image having the second image plane;
determine a second offset from center of the anatomical structure depicted in the second ultrasound image; and
provide feedback for positioning the ultrasound device based on the first and second offsets from center.

12. The apparatus of claim 11, wherein the processing device is configured, when providing the feedback for positioning the ultrasound device based on the first and second offsets from center, to provide feedback for positioning the ultrasound device to minimize the first and second offsets from center.

13. The apparatus of claim 7, wherein the processing device is configured, when providing the feedback for centering an anatomical structure depicted in the one or more first ultrasound images having the first image plane and the one or more second ultrasound images having the second image plane, to provide feedback for centering an anatomical structure depicted in the one or more first ultrasound images having the first image plane and subsequently providing feedback for centering the anatomical structure depicted in the one or more second ultrasound images having the second image plane.

14. The apparatus of claim 13, wherein the processing device is configured, when providing the feedback for centering the anatomical structure depicted in the one or more first ultrasound images having the first image plane and subsequently providing the feedback for centering the anatomical structure depicted in the one or more second ultrasound images having the second image plane, to:
receive a first ultrasound image having the first image plane;
determine a first offset from center of the anatomical structure depicted in the first ultrasound image;
provide feedback for positioning the ultrasound device based on the first offset from center; and
subsequent to providing the feedback for positioning the ultrasound device based on the first offset from center:
receive a second ultrasound image having the second image plane;
determine a second offset from center of the anatomical structure depicted in the second ultrasound image; and
provide feedback for positioning the ultrasound device based on the second offset from center.

15. The apparatus of claim 14, wherein the processing device is configured, when providing the feedback for positioning the ultrasound device based on the first offset from center, to provide feedback for positioning the ultrasound device to minimize the first offset from center.

16. The apparatus of any of claims 14, wherein the processing device is configured, when providing the feedback for positioning the ultrasound device based on the second offset from center, to provide the feedback subsequent to determining that the first offset from center is within a threshold of zero.

17. The apparatus of claim 1, wherein the feedback is of an implicit type.

18. The apparatus of claim 1, wherein the feedback is of an explicit type.

19. The apparatus of claim 1, wherein the first image plane and the second image plane are orthogonal to each other.

20. The apparatus of claim 1, wherein:
the first image plane is along an azimuthal dimension of a transducer array of the ultrasound device, and the second image plane is along an elevational dimension of the transducer array of the ultrasound device; or
the first image plane is along an elevational dimension of a transducer array of the ultrasound device, and the second image plane is along an azimuthal dimension of the transducer array of the ultrasound device.
